(19) United States
(12) Patent Application Publication
(10) Pub. No.: US 2003/0221206 A1
(43) Pub. Date: Nov. 27, 2003

(54) Methods for producing transgenic animals

(75) Inventors: Gerald Schatten, Portland, OR (US); Anthony W.S. Chan, Aloha, OR (US)

Correspondence Address: KLARQUIST SPARKMAN, LLP, 121 SW SALMON STREET, SUITE 1600, PORTLAND, OR, 97204, US

(73) Assignee: Oregon Health & Science University

(21) Appl. No.: 10/421,357

(22) Filed: Apr. 21, 2003

Related U.S. Application Data
(63) Continuation of application No. 09/736,271, filed on Dec. 15, 2000, now abandoned.
(60) Provisional application No. 60/172,142, filed on Dec. 17, 1999.

(51) Int. Cl. A01K 67/027; C12N 15/861

(52) U.S. Cl. 800/14; 800/19; 800/20; 119/300; 435/320.1; 435/456

(57) ABSTRACT

The present invention relates to methods for producing transgenic animals. Specifically, the methods of the present invention include production of a transgenic animal by transgenic intracytoplasmic sperm injection, retroviral gene transfer, intracytoplasmic nuclear injection, and pronuclear injection. In addition, the present invention also relates to methods for using transgenic animals as models for human disease and diagnosis. In particular, these transgenic animals may be used as models for embryo and fetal development, as models to assess the safety and efficacy of drug therapy and gene therapy, and as models for disease diagnosis. The methods of the present invention are also directed to methods of using transgenic embryonic cells to treat human diseases.

### FIELD OF THE INVENTION

The present invention relates to methods for producing transgenic animals and methods for using said transgenic animals as models for human disease and diagnosis.

### BACKGROUND OF THE INVENTION

Numerous transgenic animals have been created in the development of transgenic technology (Palmiter et al., 300 NATURE 611-15, 1982; Ebert et al., 2 MOL. ENDOCRIN. 277-83, 1988; Sutrave et al., 4 GENE DEV. 1462-72, 1990; Pursel et al., 45 THERIOGENOLOGY 348, 1996). For example, transgenic animals have been developed to serve as bioreactors for the production of pharmaceuticals (Clark et al., 7 BIOTECH. 487-92, 1989; Wilmut et al., 41 J. REPROD. FERT. 135-46, 1990; Krimpenfort et al., 9 BIOTECH. 844-47, 1991; Schnieke et al., 278 SCIENCE 2130-33, 1997). This technology has been focused primarily on the production of transgenic mice (see e.g., U.S. Pat. Nos. 6,137,029; 6,156,727; 6,127,598; 6,111,166; 6,107,541; and 6,077,990).

Transgenic animals have provided models for human diseases resulting in new molecular maps of metabolic processes (Nishimori and Matzuk, 1 REV. REPROD. 203-12, 1996). While most of these investigations have been performed using transgenic mice, studies are now emerging on other transgenic animals, demonstrating a wealth of biomedical, pharmaceutical (i.e., “pharming”), and agricultural implications (see e.g., U.S. Pat. No. 6,147,202). Notwithstanding the powerful technologies now available for creating rodent models for various diseases, these models are not always appropriate in studying human disorders. Extending transgenesis approaches to nonhuman primates will further enhance the utility of this model. The production of transgenic nonhuman primates as clinically relevant models for human disease is of vital importance for biomedical research. Furthermore, the promise of safe and effective gene therapy protocols cannot be fully realized until an appropriate system for investigation is found to fill the gap between knockout mice and seriously ill patients. Moreover, the similarities between nonhuman primate and humans enhance the utility of transgenic methods for devising models for testing the safety and efficacy of emerging gene therapy approaches. Consequently, there is a need for reliable and effective methods for producing genetically modified nonhuman primates.

The creation of a transgenic nonhuman primate has proven to be a difficult task. This is due, in part, to: the limited number of monkeys available as oocyte donors; the scarcity of properly staged surrogates; the limited number of embryos developing to the blastocyst stage for selection of the transgenic embryos for possible transfer; the lack of optimized procedures for successful nonsurgical embryo transfer beyond the 4- to 8-cell stage (i.e., either just prior to or at the time of the maternal to embryonic transition); and, the high cost of each experiment. Additionally, a major obstacle in producing transgenic nonhuman primates has been the low efficiency of conventional gene transfer protocols.

The present invention provides improved methods for the generation of transgenic animals. In particular, the present invention relates to methods for the production of transgenic nonhuman primates. These methods may provide the means for creating genetically modified nonhuman primates invaluable for studies across the entire spectrum of biomedical research, e.g., aging, AIDS, cancer, Alzheimer's disease, autoimmune diseases, metabolic disorders, and obesity. Additional applications of transgenesis include the production of models for investigating the molecular basis of hereditary diseases, demonstration of the safety and efficacy of gene, stem or somatic cell therapy prior to clinical trials, endangered species preservation, and perhaps even a new approach for gamete-mediated gene therapy.

### SUMMARY OF THE INVENTION

The present invention is directed to methods for producing a transgenic animal by transferring exogenous DNA from spermatazoa to oocytes by intracytoplasmic sperm injection (ICSI). In a preferred embodiment, the oocytes are cultured to an embryonic stage, the embryos are then transferred to surrogate females, and subsequently, a transgenic animal is produced by parturition. In another embodiment of the present invention, the oocyte is cultured to the 3-16 cell embryo stage. In a further aspect of the present invention, the exogenous DNA is bound to spermatozoa by mixing the exogenous DNA with spermatozoa; incubating DNA-spermatozoa mixture for 30 minutes at 37° C.; and washing DNA-bound spermatozoa in TALP-HEPES buffer.

In another embodiment of the methods of the present invention, the transgenic animal may be a mammal, bird, reptile, amphibian, or fish. In another aspect of this method, the transgenic animal is a nonhuman primate. In a further preferred embodiment of the present invention, the transgenic nonhuman primate may be a rhesus macaque, baboon, capuchin, chimpanzee, pigtail macaque, sooty mangabey, squirrel monkey, orangutan, or other nonhuman primate.

In another aspect of the present invention, the exogenous DNA is an expression vector comprising regulatory nucleic acid sequences and one or more structural gene sequences. The expression vectors of the present invention may further comprise plasmid vectors, viral vectors, and retroviral vectors. In addition, the exogenous DNA may comprise of one or more expression vectors.

In a further embodiment of the present invention, the regulatory nucleic acid sequence of the expression vector is a promoter. In one aspect of the present invention, the promoter is a viral promoter, constitutive promoter, or inducible promoter. More specifically, the promoter of the present invention is the cytomegalovirus promoter. In a further aspect of the present invention, the promoter is the protamine-1 promoter.

The present invention also relates to a structural gene sequence which encodes a polypeptide selected from the group consisting of receptors, enzymes, cytokines, hormones, growth factors, immunoglobulins, cell cycle proteins, cell signaling proteins, membrane proteins, and cytoskeletal proteins.

In another aspect of the present invention, the structural gene sequence is a reporter gene. Specifically, the reporter gene is the green fluorescent protein gene or the reporter gene is selected from the group consisting of β-galactosidase gene, secreted placental alkaline phosphatase gene, and luciferase gene.

In an alternative aspect, the structural gene sequence is a disease gene. More specifically, the disease gene has been associated with a disease selected from the group consisting of cardiovascular disease, neurological diseases, reproductive disorders, cancer, eye diseases, endocrine disorders, pulmonary disease, metabolic disorders, hereditary diseases, autoimmune disorders, and aging.

In a preferred embodiment of the present invention, the exogenous DNA is labeled with a fluorophore. In another aspect, the fluorophore is rhodamine.

Also within the scope of the present invention are methods for producing transgenic animals as models for human disease. Specifically, models for human disease may be selected from the group consisting of cardiovascular disease, neurological diseases, reproductive disorders, cancer, eye diseases, endocrine disorders, pulmonary disease, metabolic disorders, autoimmune disorders, and aging. In another aspect, the methods of the present invention are used to produce transgenic animals that are models for hereditary disease, for embryo and fetal development, and for disease diagnosis. In yet another aspect of the present invention, the transgenic animal is a model to demonstrate the safety and efficacy of treatments selected from the group consisting of drug therapy, gene therapy, stem cell therapy, and somatic cell therapy.

In another embodiment, the method of the present invention is used to preserve an endangered species. In another aspect, the method is used for sperm-mediated gene therapy.

This invention also relates to transgenic embryos produced according to the method described herein. In a preferred embodiment, the transgenic embryo is a model for embryo and fetal development. In another aspect of the present invention, the transgenic embryo is a transgenic chimeric embryo.

Also within the scope of the present invention are transgenic animals produced according to the method described herein. In a preferred embodiment, the transgenic animals are models for human disease, hereditary disease, and disease diagnosis. In another aspect, the transgenic animals are used as models to demonstrate the safety and efficacy of treatments selected from the group consisting of drug therapy, gene therapy, stem cell therapy, and somatic cell therapy.

The present invention also relates to methods of sanitizing spermatozoa by chemical decontamination and physical removal. Specifically, proteinases, DNases, and RNases may be used to chemically decontaminate spermatozoa, and polystryene and magnetic beads may be used to physically remove any decontaminants.

The present invention is also directed to methods for producing a transgenic animal by transferring exogenous DNA to oocytes by injection of a retroviral vector. In a preferred embodiment, the oocytes are then fertilized by intracytoplasmic sperm injection, cultured to an embryonic stage, transferred to surrogate females, and a transgenic animal is produced by parturition. The retroviral vector is preferably injected into the perivitelline space of the oocyte. In another aspect of the present invention, the oocyte is a prematuration oocyte or prefertilization oocyte. In a further aspect, the oocyte is cultured to the 4-8 cell embryo stage.

In another embodiment, the transgenic animal is preferably a nonhuman primate. In a further embodiment of the present invention, the nonhuman primate is a rhesus macaque, baboon, capuchin, chimpanzee, pigtail macaque, sooty mangabey, squirrel monkey, orangutan, or other nonhuman primates.

In an another aspect of the present invention, the retroviral vector comprises regulatory gene sequences and structural gene sequences. The regulatory gene sequence may be a promoter, preferably a viral promoter. In one aspect of the invention, the promoter is the cytomegalovirus promoter or the human elongation factor-1 alpha promoter. In a further aspect, the retroviral vector is a Moloney murine leukemia virus, Harvey murine sarcoma virus, murine mammary tumor virus, or Rous sarcoma virus.

The present invention also relates to methods of detecting of a retroviral vector. In particular, assays such as CV-1/S+L-assay, PCR, Southern analysis, and clonal CV-1-LNC-EGFP cells may be used to detect the presence of a retroviral vector in a tissue sample.

Also within the scope of the present invention are retroviral vectors containing a membrane-associated protein. In a preferred embodiment, the membrane-associated protein is a glycoprotein selected from Rhabdoviridae. In a further aspect, the membrane-associated protein is a glycoprotein from vesicular stomatitis virus, Piry virus, Chandipura virus, Spring viremia of carp virus, Rabies virus, or Mokola virus.

The present invention also relates to retroviral vectors comprising structural genes which encode a polypeptides selected from the group consisting of receptors, enzymes, cytokines, hormones, growth factors, immunoglobulins, cell cycle proteins, cell signaling proteins, membrane proteins, and cytoskeletal proteins.

In another aspect, the structural gene of the retroviral vector is a reporter gene. Specifically, the reporter gene may be selected from the group consisting of green fluorescent protein gene, β-galactosidase gene, secreted placental alkaline phosphatase gene, and luciferase gene.

The present invention is also directed to methods of producing a transgenic primate by intracytoplasmic nuclear injection. In a preferred embodiment, blastomeres are dissociated from an embryo and nuclei are isolated from the blastomeres. The blastomere nuclei are injected into an enucleated oocyte by intracytoplasmic nuclear injection and then the oocyte is activated. Following activation, the oocyte is cultured to the embryonic stage, the embryos are then transferred to the oviduct of surrogate females, and a transgenic animal is produced by parturition. In a further aspect, inner cell mass cells are isolated from said blastomere for nuclear transfer.

In another embodiment of the intracytoplasmic nuclear injection method, the transgenic animal is preferably a nonhuman primate. In a further embodiment of the present invention, the nonhuman primate is a rhesus macaque, baboon, capuchin, chimpanzee, pigtail macaque, sooty mangabey, squirrel monkey, orangutan, or nonhuman primates.

In further aspect of the intracytoplasmic nuclear injection method, oocyte activation is accomplished by chemical activation, sperm cytosolic (oscillin) activation, or electrical activation.

Also within the scope of the present invention are methods of producing a transgenic primate by intracytoplasmic nuclear injection using nuclei isolated from somatic cells, preferably skin cells.

The methods of the present invention also relate to methods for producing a transgenic primate by pronuclear injection. Preferably, an oocyte is fertilized by intracytoplamic sperm injection. In a further aspect, the exogenous DNA is transferred to the pronucleus of the fertilized zygote by pronuclear injection. In a preferred embodiment, the zygote is cultured to the embryo stage, the embryo is then transferred to oviduct of surrogate females, and a transgenic animal is produced by parturition.

A further aspect of the present invention are methods of using transgenic embryonic cells to treat human diseases. Specifically, the methods to produce transgenic animals and transgenic primates described in the present invention, may also be used to create transgenic embryonic stem cells. These transgenic embryonic cells may then be used to treat diseases such as cardiovascular disease, neurological diseases, reproductive disorders, cancer, eye diseases, endocrine disorders, pulmonary disease, metabolic disorders, autoimmune disorders, and aging.

### BRIEF DESCRIPTION OF THE DRAWINGS

FIGS. 1A-1H. Plasmid transfer by ICSI (FIGS. 1A-1H). Rhodamine-labeled plasmid DNA binds avidly to mouse (1A), bovine (1B), and rhesus sperm (1C). Rhodamine-tagged DNA remains on the surface of microinjected sperm after ICSI: rhesus sperm microinjected into a rhesus oocyte (1D) or into a bovine oocyte (1E). Detection of GFP expression in a 16-cell stage rhesus embryo (1F) using anti-GFP monoclonal antibody and Hoechst DNA staining. Live 4-cell (1G) and blastocyst stage (1H) rhesus monkey embryos expressing GFP after transgenesis by ICSI using rhodamine-labeled plasmid DNA encoding the GFP gene bound to the injected sperm. FIGS. 1A-1E were collected by laser scanning confocal microscopy. FIGS. 1A, 1B, and 1C were produced by overlaying images of 14 labeled sperm and each individual image of sperm is an overlay of 16 images taken at different focal planes. FIG. 1F was collected by digital lowlight level fluorescence imaging (Princeton CCD, Differential interference contrast, Zeiss Axiophot).

FIGS. 2A-2C. Live, digital lowlight level epifluorescence imaging of rhesus ICSI using sperm bound with rhodamine-labeled plasmid DNA (FIGS. 2A-2C). A single sperm, suspended in 10% PVP and displaying rhodamine labeling, is aspirated tail-first into an injection pipette (2A). The pipette is inserted through the zona and oolemma membrane of an oocyte, immobilized with a second suction pipette, and the sperm is placed deep within the oocyte cytoplasm (2B). A brief aspiration of cytoplasm ensures the correct positioning of the sperm within the oocyte prior to its release (2C). All procedures are performed at 100× magnification using digital lowlight level fluorescence imaging to ensure continued rhodamine visualization.

FIGS. 3A-3E. Injection of VSV-G pseudotyped retroviral vector, which carries GFP protein in the vector particles, into the perivitelline space (PVS) of mature rhesus oocytes (FIGS. 3A-3E). Injection of vector solution into the PVS, (3A) transmission light and (3B) fluorescence with FITC filter set. Rhesus oocytes after PVS injection of vector, (3C) transmission light and (3D) fluorescence. At 4.5 hours, vector particle can be found inside the oocyte cytoplasm (arrow, 3E).

FIGS. 4A-4L PCR and RT-PCR analysis of tissues retrieved from stillborn fetuses (FIGS. 4A-4I). A total of 13 tissues from an intact fetus were submitted for PCR analysis (4A) and 11 tissues for RT-PCR analysis (4B). Overall analysis of intact fetus was presented in (4C). Tissues from a reabsorbed fetus were collected from eight different regions to ensure broad representation, since precise anatomical specification was limited. PCR, RT-PCR, and overall analysis of the reabsorbed fetus were demonstrated in (4D, 4E, and 4F). Pl-placenta; Lu-lung; Li-liver; He-heart; In-intestine; Ki-kidney; Bl-bladder; Te-testis; Mu-muscle; Sk-skin; Ta-tail; Pa-pancreas; Sp-spleen; T1-placenta from reabsorbed fetus; T2-T9: tissues retrieved from eight regions of the reabsorbed fetus; C1-non-transgenic rhesus tissue; C2-C1+pLNC-EGFP; C3-ddH₂O; C4-293GP-LNCEGFP packaging cell; C5-non-transgenic liver; C6-transgenic lung without DNase; C7-transgenic lung without reverse transcription. A total of 7 samples from each offspring were obtained for PCR analysis (4G) and 2 samples for RT-PCR analysis (4H) from the babies (“ANDi” and Monkey B). Analysis of the newborns (4I), indicates that “ANDi” is a transgenic male with the presence of mRNA in all analyzed tissues. Pl-placenta; Cd-cord; Bl-whole blood; Ly-lymphocyte; Bu-buccal smear; Ur-urine; and Ha-hair.

FIGS. 5A-5E. The expression of GFP reporter (FIGS. 5A-5E). GFP expression in stillborn fetuses was observed in both hair shaft (5A) and toenail (5B) by direct fluorescent examination. Immuno-staining and epifluorescent examination of placenta frozen section demonstrate the presence of GFP protein. Immunostaining using anti-GFP monoclonal antibody and secondary antibody conjugated with rhodamine (5C). Epifluorescence of the same section demonstrates the expression of GFP protein (5D). The co-localization of GFP proteins (arrows) by overlaying images of immunostaining and epifluorescence (5E). Nucleus was stained using Hoechst DNA staining.

FIGS. 6A-6D. Southern blot analysis of Hind III (single digestion site) digested genomic DNA (6A). Full-length GFP labeled with ³²P was used as a probe to detect the transgene, which was detected in genomic DNA of a normal male stillbirth (6B) and a reabsorbed fetus (6C). Non-transgenic rhesus tissue was used as a negative control and pLNC-EGFP DNA as a positive control. Various sized fragments were demonstrated in tissues obtained from each. This result indicates multiple integration sites due to the use of a restriction enzyme with a single digestion site within the transgene. Detection of the unique provirus sequence (6D). A total of 5 tissues from each infant and two tissues from a male stillbirth and a reabsorbed fetus were submitted for PCR. Provirus sequence was detected in “ANDi” and the two stillbirths, which indicates that they are transgenic. Abbreviations are the same as FIGS. 4A-4I. Mu-Muscle from the intact fetus and T3-tissue from the reabsorbed fetus.

### DETAILED DESCRIPTION OF THE INVENTION

Before the methods for producing transgenic animals are described in the present invention, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Definitions

For convenience, the meaning of certain terms and phrases employed in the specification, examples, and appended claims are provided below.

As used herein, the term “egg” when used in reference to a mammalian egg, means an oocyte surrounded by a zona pellucida and a mass of cumulus cells (follicle cells) with their associated proteoglycan.

The term “oocyte” refers to a female gamete cell and includes primary oocytes, secondary oocytes and mature, unfertilized ovum. An oocyte is a large cell having a large nucleus (i.e., the germinal vesicle) surrounded by ooplasm. The ooplasm contains non-nuclear cytoplasmic contents including mRNA, ribosomes, mitochondria, yolk proteins, etc.

The term “prefertilization oocyte” as used herein refers to a female gamete cell such as a pre-maturation oocyte following exposure to maturation medium in vitro but prior to exposure to sperm (i.e., matured but not fertilized). The prefertilization oocyte has completed the first meiotic division, has released the first polar body and lacks a nuclear membrane (the nuclear membrane will not reform until fertilization occurs; after fertilization, the second meiotic division occurs along with the extrusion of the second polar body and the formation of the male and female pronuclei). Prefertilization oocytes may also be referred to as matured oocytes at metaphase II of the second meiosis.

The terms “unfertilized egg” or “unfertilized oocyte” as used herein refers to any female gamete cell which has not been fertilized and these terms encompass both pre-maturation and pre-fertilization oocytes.

The term “perivitelline space” refers to the space located between the zona pellucida and the plasma membrane of a mammalian egg or oocyte.

The term “sperm” refers to a male gamete cell and includes spermatogonia, primary spermatocytes, secondary spermatocytes, spermatids, differentiating spermatids, round spermatids, and spermatozoa.

The term “somatic cell” refers to any animal cell other than a germ cell or germ cell precursor.

The term “embryonic stem cell” or “stem cell” refers a cell which is an undifferentiated cell and may undergo terminal differentiation giving rise to many differentiated cell types in an embryo or adult, including the germ cells (sperm and eggs). This cell type is also referred to as an “ES cell” herein.

The term “animal” includes all vertebrate animals such as mammals (e.g., rodents, primates (e.g., monkeys, apes, and humans), sheep, dogs, cows, pigs), amphibians, reptiles, fish, and birds. It also includes an individual animal in all stages of development, including embryonic and fetal stages.

A “transgenic animal” refers to any animal, preferably a mammal (e.g., mouse, rat, squirrel, hamster, guinea pig, pig, baboons, squirrel monkey, and chimpanzee, etc.), bird or an amphibian, in which one or more cells contain heterologous nucleic acid introduced by way of human intervention. The transgene is introduced into the cell, directly or indirectly, by introduction into a precursor of the cell, by way of deliberate genetic manipulation, or by infection with a recombinant virus. In the transgenic animals described herein, the transgene causes cells to express a structural gene of interest. However, transgenic animals in which the transgene is silent are also included.

The term “transgenic cell” refers to a cell containing a transgene.

The term “germ cell line transgenic animal” refers to a transgenic animal in which the genetic alteration or genetic information was introduced into a germ line cell, thereby conferring the ability to transfer the genetic information to offspring. If such offspring in fact possess some or all of that alteration of genetic information, they are transgenic animals as well.

The term “gene” refers to a DNA sequence that comprises control and coding sequences necessary for the production of a polypeptide or precursor. The polypeptide can be encoded by a full length coding sequence or by any portion of the coding sequence so long as the desired enzymatic activity is retained.

The term “transgene” broadly refers to any nucleic acid that is introduced into the genome of an animal, including but not limited to genes or DNA having sequences which are perhaps not normally present in the genome, genes which are present, but not normally transcribed and translated (“expressed”) in a given genome, or any other gene or DNA which one desires to introduce into the genome. This may include genes which may normally be present in the nontransgenic genome but which one desires to have altered in expression, or which one desires to introduce in an altered or variant form. The transgene may be specifically targeted to a defined genetic locus, may be randomly integrated within a chromosome, or it may be extrachromosomally replicating DNA. A transgene may include one or more transcriptional regulatory sequences and any other nucleic acid, such as introns, that may be necessary for optimal expression of a selected nucleic acid. A preferred transgene of the invention is a viral transgene. A transgene can be as few as a couple of nucleotides long, but is preferably at least about 50, 100, 150, 200, 250, 300, 350, 400, or 500 nucleotides long or even longer and can be, e.g., an entire viral genome. A transgene can be coding or non-coding sequences, or a combination thereof. A transgene usually comprises a regulatory element that is capable of driving the expression of one or more transgenes under appropriate conditions.

The phrase “a structural gene of interest” refers to a structural gene which expresses a biologically active protein of interest or an antisense RNA for example. The term “structural gene” excludes the non-coding regulatory sequence which drives transcription. The structural gene may be derived in whole or in part from any source known to the art, including a plant, a fungus, an animal, a bacterial genome or episome, eukaryotic, nuclear or plasmid DNA, cDNA, viral DNA, or chemically synthesized DNA. A structural gene may contain one or more modifications in either the coding or the untranslated regions which could affect the biological activity or the chemical structure of the expression product, the rate of expression, or the manner of expression control. Such modifications include, but are not limited to, mutations, insertions, deletions, and substitutions of one or more nucleotides. The structural gene may constitute an uninterrupted coding sequence or it may include one or more introns, bound by the appropriate splice junctions. The structural gene may also encode a fusion protein.

The term “heterologous DNA,” which is used interchangeably with “exogenous DNA” refers to DNA that is not naturally present in the cell.

The term “genome” is intended to include the entire DNA complement of an organism, including the nuclear DNA component, chromosomal or extrachromosomal DNA, as well as the cytoplasmic domain (e.g., mitochondrial DNA).

The term “transgene construct” refers to a nucleic acid molecule, (e.g., vector), which contains a structural gene of interest that has been generated for the purpose of the expression of a specific nucleotide sequence(s), or is to be used in the construction of other recombinant nucleotide sequences.

As used herein, the term “vector” refers to a nucleic acid molecule capable of transporting another nucleic acid to which it has been linked. Preferred vectors are those capable of autonomous replication and/expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer to circular double-stranded DNA that in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

“Gene expression” refers to the process by which a nucleotide sequence undergoes successful transcription and translation such that detectable levels of the delivered nucleotide sequence are expressed.

The term “promoter” refers to the minimal nucleotide sequence sufficient to direct transcription. Also included in the invention are those promoter elements that are sufficient to render promoter-dependent gene expression controllable for cell-type specific, tissue specific, or inducible by external signals or agents; such elements may be located in the 5′ or 3′ regions of the native gene, or in the introns. The term “inducible promoter” refers to a promoter where the rate of RNA polymerase binding and initiation of transcription can be modulated by external stimuli. The term “constitutive promoter” refers to a promoter where the rate of RNA polymerase binding and initiation of transcription is constant and relatively independent of external stimuli. A “temporally regulated promoter” is a promoter where the rate of RNA polymerase binding and initiation of transcription is modulated at a specific time during development.

As used herein, the term “regulatory sequence” refers to a nucleic acid sequence capable of controlling the transcription of an operably associated gene. A regulatory sequence of the invention may include a promoter, an enhancer, and/or a silencer. Therefore, placing a gene under the regulatory control of a promoter or a regulatory element means positioning the gene such that the expression of the gene is controlled by the regulatory sequence(s). In general, promoters are found positioned 5′ (upstream) of the genes that they control. Thus, in the construction of promoter gene combinations, the promoter is preferably positioned upstream of the gene and at a distance from the transcription start site that approximates the distance between the promoter and the gene it controls in the natural setting. As is known in the art, some variation in this distance can be tolerated without loss of promoter function. Similarly, the preferred positioning of a regulatory element, such as an enhancer, with respect to a heterologous gene placed under its control reflects its natural position relative to the structural gene it naturally regulates. Enhancers are believed to be relatively position and orientation independent in contrast to promoter elements. In addition, 3′ untranslated regions such as polyA signals may also be utilized as a regulatory sequence.

The term “antisense nucleic acid” refers to nucleic acid molecules (e.g., molecules containing DNA nucleotides, RNA nucleotides, or modifications (e.g., modifications that increase the stability of the molecule, such as 2′-O-alkyl (e.g., methyl) substituted nucleotides) or combinations thereof) that are complementary to, or that hybridize to, at least a portion of a specific nucleic acid molecule, such as an RNA molecule (e.g., an mRNA molecule). The antisense nucleic acids hybridize to corresponding nucleic acids, such as mRNAs, to form a double-stranded molecule, which interferes with translation of the mRNA, as the cell will not translate a double-stranded mRNA. Antisense nucleic acids used in the invention are typically at least 10-12 nucleotides in length, for example, at least 15, 20, 25, 50, 75, or 100 nucleotides in length. The antisense nucleic acid can also be as long as the target nucleic acid with which it is intended to form an inhibitory duplex. The antisense nucleic acids can be introduced into cells as antisense oligonucleotides, or can be produced in a cell in which a nucleic acid encoding the antisense nucleic acid has been introduced.

The term “retroviral vector” refers to a retrovirus or retroviral particle which is capable of entering a cell and integrating the retroviral genome (as a double-stranded provirus) into the genome of the host cell.

Transgenic animal models for human diseases have lead to remarkable breakthroughs, revealing the molecular basis of numerous illnesses. These discoveries are already influencing disease diagnosis, treatment and even cures (Palmiter et al., 300 NATURE 611-15, 1982; Koopman et al., 351 NATURE 117-121, 1991; Wright et al., 9 BIOTECH. 330-34, 1991; Tang et al., 49 BIOL. REPROD. 346-53, 1993). Biomedical researchers have chosen the transgenic mouse model for several reasons, including: the wealth of knowledge in preparation the gametes, embryos, and surrogates; cost and availability; short generation time; numerous inbred strains displaying particularly useful markers and/or features; and a large genetic database (HOGAN ET AL., MANIPULATION OF THE MOUSE EMBRYO, Cold Spring Harbor Press, Long Island, N.Y., 1986). Although the transgenic mouse model provides a valuable tool, there are questions that cannot be adequately answered in mice due to their differences from humans. Consequently, there is a need to develop and optimize an innovative approach for creating transgenic nonhuman primates.

The present invention provides for transgenic animals that carry the transgene in all their cells, as well as animals that carry the transgene in some, but not all cells, i.e., mosaic animals. The transgene can be integrated as a single transgene or in tandem, e.g., head to head tandems, or head to tail, or tail to tail, or as multiple copies. Double, triple, or multimeric transgenic animals may preferably comprise at least two or more transgenes. In a preferred embodiment, the animal comprises the GFP transgene and a transgene encoding a structural gene of interest.

Where one or more genes encoding a protein are used as transgenes, it may be desirable to operably link the gene to an appropriate regulatory element, which will allow expression of the transgene. Regulatory elements, e.g., promoters, enhancers, (e.g., inducible or constitutive), or polyadenylation signals are well known in the art. Regulatory sequences can be endogenous regulatory sequences, i.e., regulatory sequences from the same animal species as that in which it is introduced, as a transgene. The regulatory sequences can also be the natural regulatory sequence of the gene that is used as a transgene.

A transgene construct described herein may include a 3′ untranslated region downstream of the DNA sequence. Such regions can stabilize the RNA transcript of the expression system and thus increase the yield of desired protein from the expression system. Among the 3′ untranslated regions useful in the constructs of this invention are sequences that provide a polyA signal. Such sequences may be derived, e.g., from the SV40 small t antigen, or other 3′ untranslated sequences well known in the art. The length of the 3′ untranslated region is not critical but the stabilizing effect of its polyA transcript appears important in stabilizing the RNA of the expression sequence.

A transgene construct may also include a 5′ untranslated region between the promoter and the DNA sequence encoding the signal sequence. Such untranslated regions can be from the same control region from which promoter is taken or can be from a different gene, e.g., they may be derived from other synthetic, semi-synthetic, or natural sources.

Antisense nucleic acids may also be used in the transgene construct of the present invention. For example, an antisense polynucleotide sequence (complementary to the DNA coding strand) may be introduced into the cell to decrease the expression of a “normal” gene. This approach utilizes, for example, antisense nucleic acid, ribozymes, or triplex agents to block transcription or translation of a specific mRNA, either by masking that mRNA with an antisense nucleic acid or triplex agent, or by cleaving it with a ribozyme. Alternatively, the method includes administration of a reagent that mimics the action or effect of a gene product or blocks the action of the gene. The use of antisense methods to alter the in vitro translation of genes is well known in the art (see e.g., Marcus-Sekura, 172 ANAL. BIOCHEM. 289-95, 1988).

The transgene constructs described herein may be inserted into any suitable plasmid, bacteriophage, or viral vector for amplification, and may thereby be propagated using methods known in the art, such as those described by Maniatis et al. (MOLECULAR CLONING: A LABORATORY MANUAL, Cold Spring Harbor, N.Y., 1989). A construct may be prepared as part of a larger plasmid, which allows the cloning and selection of the constructions in an efficient manner as is known in the art. Constructs may be located between convenient restriction sites on the plasmid so that they may be easily isolated from the remaining plasmid sequences for incorporation into the desired mammal.

Another embodiment of the present invention provides a method for producing transgenic animals, preferably nonhuman primates, by the introduction of exogenous DNA via pronuclear injection (BREM AND MULLER, ANIMALS WITH NOVEL GENES, Cambridge University Press (N. Maclean, ed.) 179-244, 1994; Wall, 45 THERIOGENOLOGY 57-68, 1996). The pronuclei are formed by the decondensation of the gamete nuclei following incorporation of the spermatazoa into the cytoplasm of the oocyte. The direct injection of DNA into the pronucleus produces a localized increased concentration of DNA which facilitates intramolecular and intermolecular associations resulting in DNA insertion at a chromosomal breakage point and subsequent DNA repair (Bishop, 36 REPROD. NUTR. DEV. 607-18, 1996). Pronuclear injection usually results in multiple transgene copies at a single insertion site. The insertion of the exogenous DNA into the chromosome most likely occurs during DNA replication (Coffin, 31 J. MED. VIROL. 43-49, 1990). The size of the DNA fragment used in this technique may be quite large (Brem et al., 44 MOL. REPROD. DEV. 56-62, 1996). Thus, to control the expression of the transgene, regulatory elements such as a locus control region or centromeric region may be included in the exogenous DNA. For transgenic primates, pronuclear injection may be an efficient way to create transgenic embryos since both pronuclei are readily visible.

Generally, the transgene is introduced by microinjection and the fertilized oocytes are then cultured in vitro until a pre-implantation embryo is obtained preferably containing about 16-150 cells (see e.g., U.S. Pat. No. 4,873,191). Methods for culturing fertilized oocytes to the pre-implantation stage are described by Gurdon et al. (101 METH. ENZYMOL. 370-86, 1984); HOGAN ET AL. (MANIPULATION OF THE MOUSE EMBRYO: A LABORATORY MANUAL, C.S.H.L. N.Y., 1986); Hammer et al. (315 NATURE 680-83, 1985); Gandolfi et al. (81 J. REPROD. FERT. 23-28, 1987); Rexroad et al. (66 J. ANIM. SCI. 947-953, 1988); Eyestone et al. (85 J. REPROD. FERT. 715-720, 1989); and Camous et al. (72 J. REPROD. FERT. 779-785, 1984). The pre-implantation embryos may be frozen pending implantation. Pre-implantation embryos are transferred to the oviduct of a pseudopregnant female resulting in the birth of a transgenic or chimeric animal, depending upon the stage of development when the transgene is integrated. Chimeric mammals can be bred to form true germline transgenic animals.

A further aspect of the present invention is the transgenic intracytoplasmic nuclear injection (ICNI) method. ICNI is similar to nuclear transfer using electrofusion in that either an embryonic or somatic nucleus, and its associated cellular components, are transferred into an enucleated oocyte. It differs from electrofusion in several ways since the nucleus is directly injected into the oocyte cytoplasm. ICNI offers several advantages over electrofusion, particularly when working with limited numbers of oocytes. The route for nuclear injection is more controlled, and the possibility of transferring the nucleus to a particular cytoplasmic site (i.e., cortical vs. central cytoplasm) exists. Furthermore, the time of nuclear introduction can be differentiated from the time of oocyte activation. ICNI using somatic nuclei holds promise for propagating animal models with particular mutations and also for propagating identical research specimens for vaccine and physiological studies (Biggers, 26 THERIOGENOLOGY 1-25, 1986).

Prior to transfer of a diploid nucleus, genomic DNA complement has to be removed from the recipient cytoplast (mature oocyte). Efficiency of enucleation procedure prior to nuclear transfer is of crucial importance to avoid ploidy abnormalities with its detrimental effects on later embryonic development, to eliminate any genetic contribution of the recipient cytoplasm, and for excluding the possibility of parthenogenetic activation and embryo development without the participation of the newly introduced nucleus. Enucleation has been accomplished successfully in a range of species by labeling oocyte DNA with Hoechst 33342 (Critser and First, 61 STAIN TECHNOL. 1-5, 1986; Smith 99 J. REPROD. FERT. 39-44, 1993). DNA labeled with the fluorochrome emits strong fluorescence when excited with ultraviolet light. DNA can therefore be visualized during the enucleation procedure ensuring its complete removal (metaphase II plate and the first polar body). A report in cattle has shown that exposure of oocytes to UV irradiation for 10 seconds has no effect on embryo viability and production of live calves (Westhusin et al., 95 J. REPROD. FERT. 475-480, 1992). Similarly, irradiation of rabbit and Xenopus oocytes for periods shorter than 15 seconds showed no effect on oocytes' developmental ability (Yang et al., 27 MOL. REPROD. DEV. 118-29, 1990; Gurdon 101 J. MICROSCOPIC SOC. 299-311, 1960). However, exposure of oocytes to UV light for 30 seconds or more causes a loss in membrane integrity, decreased methionine incorporation and significantly alters the pattern of protein synthesis in bovine oocytes (Smith, 1993), decreases viability in rabbit oocytes (Yang et al., 1990) and causes abnormal development in 30% of irradiated Xenopus oocytes (Gurdon, 1960). However, possibility of damaging effects of ultraviolet light on oocyte cytoplasm even for very short periods of time needs to be considered.

To produce enucleated unfertilized oocytes, microfilament inhibitors such as cytochalasins (B and D), colcemid, and demicolcine have been widely used for enucleation of many species (McGrath and Solter, 226 SCIENCE 1317-19, 1984; Prather et al., 37 BIOL. REPROD. 859-66, 1987; Cheong et al., 48 BIOL. REPROD. 958-63, 1993; Chastant et al., 44 MOL. PEPROD. DEV. 423-32, 1996). Other microfilament inhibitors include latrunculin A, which disrupts microfilament organization by binding to G-actin, and jasplakinolide, a macro-cyclic peptide isolated from the marine sponge, Jaspis johnstoni (Schatten et al., 83 PROC. NATL. ACAD. SCI. USA 105-09, 1986). To verify that enucleation has successfully removed the meiotic spindle from recipient oocytes, vital green and red-fluorescent nuclei acid dyes will be utilized (Thomas et al., 56 BIOL. REPROD. 991-98, 1997). Enucleation of oocytes may also be accomplished by intracytoplasmic enucleation that involves the direct aspiration of the cytoplasm after penetration of the oolemma membrane in the absence of microfilament inhibitors. Confirmation that successful enucleation of the recipient oocyte has occurred may be performed by the fluorescent analysis of the removed material. Visualization of two distinct DNA complements inside the enucleation pipette (metaphase plate and the first polar body) indicates removal of the recipient nuclear genome and prevents the need for oocyte excitation.

The methods of the present invention also relate to the production of transgenic animals by the introduction of exogenous DNA into an oocyte using retroviral vectors. Retroviral vectors can be used to transfer genes efficiently into host cells by exploiting the viral infectious process (Kim et al., 4 ANIM. BIOTECHNOL. 53-69, 1993; Kim et al., 35 MOL. REPROD. DEV. 105-13, 1993; Haskell and Bowen, 40 MOL. REPROD. DEV. 386-90, 1995; Chan et al., 95 PROC. NATL. ACAD. SCI. USA 14028-33, 1998; Krimpenfort et al., 1991; Bowen et al., 50 BIOL. REPROD. 664-68, 1994; Tada et al., 1 TRANSGENICS 535-40, 1995). Foreign or heterologous genes cloned (i.e., inserted using molecular biological techniques) into the retroviral genome can be delivered efficiently to host cells which are susceptible to infection by the retrovirus. Through well-known genetic manipulations, the replicative capacity of the retroviral genome can be destroyed. The resulting replication-defective vectors can be used to introduce new genetic material to a cell but they are unable to replicate. A helper virus or packaging cell line can be used to permit vector particle assembly and egress from the cell. The host range of a retroviral vector (i.e., the range of cells that these vectors can infect) can be altered by including an envelope protein from another closely related virus. Methods for using retroviruses for the production of transgenic animals are described in Chan et al., 1998 and U.S. Pat. No. 6,080,912.

Replication-defective retroviral vectors have been established as an efficient and safe route for gene transfer into mammalian cells (Shimotohno and Temin, 26 CELL 67-77, 1981; Rubenstein et al., 83 PROC. NATL. ACAD. SCI. USA 366-68, 1986). Genes transferred by means of retroviral infection seldom rearrange or have multiple insertions which commonly occurs with pronuclear injection (Bishop and Smith, 6 MOL. BIOL. MED. 283-98, 1989; Wall, 1996). Several studies have indicated the possible use of replication defective retroviral vectors as a medium to transfer DNA into early stage bovine embryos for the production of transgenic bovine (Kim et al., 1993; Haskell and Bowen, 1995; Chan et al., 1998). Replication-defective retroviral vectors derived from Moloney murine leukemia virus (MoMLV) can transfer foreign genes into mammalian cells efficiently (Gilboa et al., 4 BIOTECH. 504-12, 1986; Kim et al., 1993).

Integration of the retrovirus into the host cell genome is mediated by retroviral integrase and specific nucleotide sequences located at the ends of the retroviral genome (Goff, 26 ANNU. REV. GENET. 527-44, 1992). In addition, the breakdown of the nuclear envelope during mitotic M-phase is also critical for retroviral integration (Roe et al., 12 EMBO J. 2099-2108, 1993). Nuclear envelope breakdown permits the translocation of the retroviral preintegration complex into the nucleus prior to integration. During metaphase II (MII) of the second meiosis, oocytes do not possess a nuclear envelope until the formation of the pronucleus during interphase. Thus, retroviral infection of MII oocytes resulted in an enhanced gene integration efficiency in the genome (Chan et al., 1998).

Several disadvantages of conventional retroviral vectors restrict their use in targeting tissues and organs in vivo (Adam et al., 89 PROC. NATL. ACAD. SCI. USA 8981-85, 1992; Burns et al., 90 PROC. NATL. ACAD. SCI. USA 8033-37, 1993; Yee et al., 43 METHODS CELL BIOL. 99-112, 1994). The low virus titer and restricted host cell range, which are related to the stability of the viral envelope protein and mechanism of host cell recognition (Albritton et al., 57 CELL 655-59, 1989; Yee et al., 1994), are major limitations. Transgenic mice, chickens, and cattle have been produced by infecting oocytes or early stage embryos with retroviral vectors (Jaenisch et al., 1975; Stewart et al., 97 J. EMBRYOL. EXP. MORPHOL. SUPPL. 263-75, 1986; Stewart et al., 6 EMBO 383-88, 1987; Chan et al., 1998). However, the major hindrances in using replication defective retroviral vectors are the limited virus titer (10⁵-10⁶ cfu/ml) and the restricted host cell specificity (Wall and Seidel, 38 THERIOGENOLOGY 337-57, 1992; Kim et al., 1993).

To overcome the low viral titer and limited host cell range, retroviral vectors may be pseudotyped with the envelope glycoprotein of the vesicular stomatitis virus (VSV-G). This glycoprotein interacts with the phospholipid components of the host cell plasma membrane. The pseudotyped vectors displayed an expanded range of infectivity and could be concentrated (10⁹-10¹⁰ cfu/ml) without a significant loss of infectivity (Chan et al., 1998). The present invention is not limited to the use of the VSV-G protein; thus, the glycoproteins of other Vesiculovirus or Lyssa viruses may be employed.

Various viral vectors which may be utilized for transgenesis include, but are not limited to, adenovirus, herpes virus, vaccinia, or preferably, an RNA virus such as a retrovirus. Examples of retroviral vectors in which a single foreign gene can be inserted include, but are not limited to, Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transgenic cells can be identified. By inserting a gene sequence (including the promoter region) of interest into a viral vector with, for example, another gene which encodes a receptor ligand on a specific target cell, the vector is now target specific. One skilled in the art can readily ascertain the specific polynucleotide sequences which can be inserted into the retroviral genome resulting in the target specific delivery of the polynucleotide.

The role of spermatazoa during fertilization involves the transfer of a haploid genome to the resultant zygote. This capacity has been exploited as an innovative strategy for the delivery of exogenous DNA for the production of transgenic animals (Lauria & Gandolfi, 36 MOL. REPROD. DEV. 255-57, 1993; Kim et al., 46 MOL. REPROD. DEV. 1-12, 1997; Chan et al., MOL. HUMAN REPROD. 26-33, 2000; Perry et al., 284 SCIENCE 1180-83, 1999).

The methods of the present invention described herein demonstrate that exogenous DNA bound to the surface of sperm is retained and transferred into the egg during intracytoplasmic sperm injection (ICSI). As described in Example 1, exogenous DNA is mixed with sperm, incubated, and washed. The DNA-bound sperm is then injected into the oocyte by the ICSI method. This technology (“TransgenICSI”) is an innovative and powerful approach for routinely producing transgenic nonhuman primate specimens for clinically relevant research and for creating transgenic primates for diagnosing, preventing, and curing human diseases.

TransgenICSI technology provides a highly efficient means of introducing foreign DNA in animals. The use of spermatozoa as a carrier to transfer foreign DNA into mouse oocytes during in vitro fertilization has provided new insights in transgenic technology (Lavitrano et al., 57 CELL 717-23, 1989). The delivery of exogenous genetic material into primate oocytes during TransgenICSI resulted in both embryonic transgene expression as well as live births, and demonstrates the feasibility of this new procedure (Chan et al., 6 MOL. HUM. REPROD. 26-33, 2000). Primate sperm bound with DNA retained its full reproductive potential for full-term offspring normal by every measurable criteria. Although primates require greater and longer-term investments and dedication, the biomedical rationales for producing transgenic primates are clear—to fill the gap between transgenic mice and human patients. However, this undertaking differs considerably from the production of transgenic rodents, which requires only weeks to achieve live births and is a more permissive system for analyzing results following humane euthanasia. Transgenic mice have been produced by ICSI with a 2 to 2.8% DNA integration rate (Perry et al., 284 NATURE 1180-83, 1999). Breaching of the sperm's plasma-membrane may have enhance transgenesis efficiency, perhaps due to increased DNA binding, internalization and/or integration.

Transgenesis by ICSI represents a promising approach for exogenous DNA transmission, and should be particularly valuable in systems in which oocytes, surrogate mothers, and the number of embryos transferred are precious and limiting, as in primates. TransgenICSI eliminates the problem of locating the male pronucleus for subsequent microinjection within the nearly opaque cytoplasm in oocytes from domestic species (i.e., pigs and cows) or when both pronuclei are indistinguishable (e.g., primates). TransgenICSI technology also avoids the pitfalls regarding the possible loss of exogenously bound DNA during in vitro fertilization.

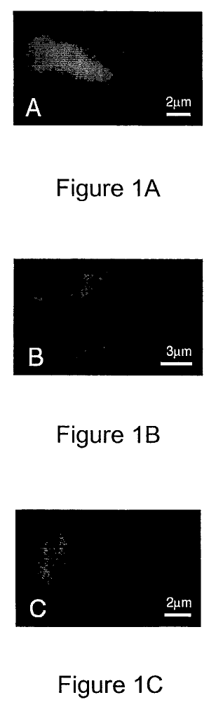

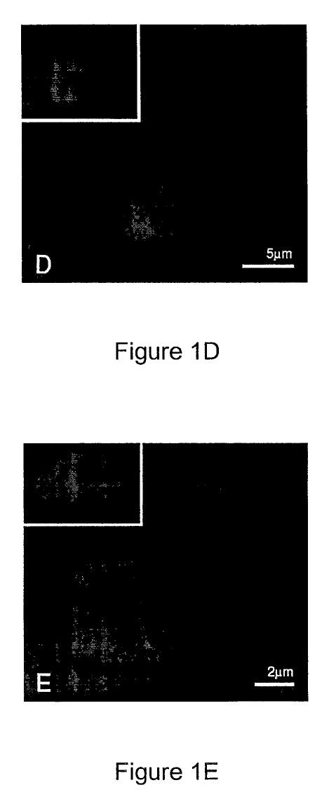

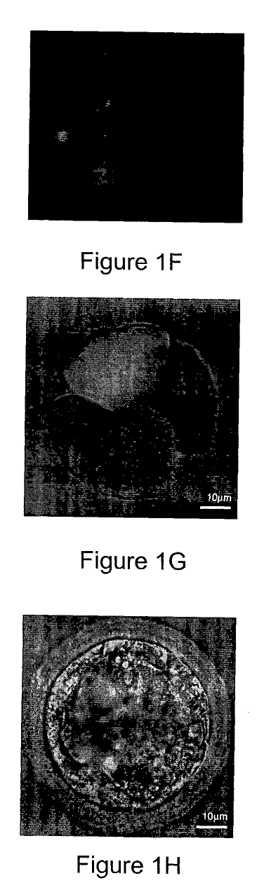

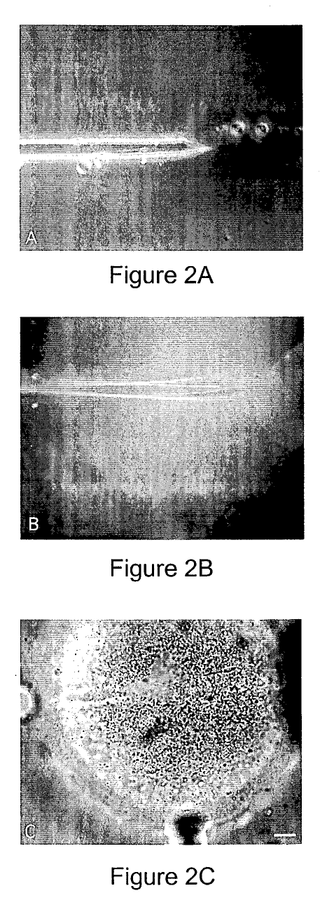

The rhodamine-tagged plasmid DNA served as a dynamic fluorescence marker that demonstrates the binding of DNA to the surface of the sperm head, as imaged with confocal microscopy (FIG. 1: mouse (A); bovine (B); rhesus (C). The rhodamine signal was retained after thorough washing, though its trypsin lability suggests that the adherence at the sperm cell surface is protein-mediated (Lavitrano et al., 31 MOL. REPROD. DEV. 161-69, 1992; Zani et al., 21 EXP. CELL RES. 57-64, 1995). Laser-scanning confocal and digital epifluorescence imaging of rhesus fertilization by ICSI demonstrated the preservation of the rhodamine-plasmid fluorescence in association with the microinjected sperm throughout the ICSI procedure and during the early stages of pronuclear development. (FIGS. 1D and 1E; FIGS. 2A-2C). The brightness of the signal, which might have been quenched by the deeper focus through the cytoplasm, indicated that most of the exogenously bound DNA is retained after ICSI. Furthermore, the plasmid remains associated with the sperm nucleus and does not disperse. Dynamic, live, high resolution imaging demonstrated the persistent binding of the rhodamine-tagged plasmid DNA to the sperm during sperm selection (FIG. 2A), during sperm microinjection (FIG. 2B), and after successful ICSI (FIG. 2C). The signal of the rhodamine-label plasmid is lost as the oocyte enters the first cell cycle because the dim image becomes undetectable as it expands and perhaps is quenched by the egg cytoplasm. Microinjected free plasmid disperses swiftly throughout the oocyte cytoplasm.

Using rhodamine-labeled plasmids encoding the green fluorescence protein (GFP), transgenic rhesus embryos expressing GFP were created at high frequency by this new approach. Rhesus sperm bound with a rhodamine-tagged plasmid encoding the GFP gene under the control of CMV promoter (Rh-CMV-GFP), retained the plasmid after microinjection into mature rhesus (FIG. 1D), or bovine (FIG. 1E) oocytes. Mosaic GFP expression is detected as early as the 4-cell stage (FIG. 1G). The number of blastomeres and the percentage of expressing embryos increase at least until the blastocyst stage, in which both the inner cell mass and trophectoderm exhibit GFP-fluorescence (FIG. 1H). Direct GFP fluorescence detection is not the most sensitive indicator of GFP expression. Although undetectable by direct GFP imaging, an embryo was fixed and labeled with anti-GFP antibody. A single blastomere with a detectable signal under fluorescent microscopy was observed, indicating that GFP expression of the transgene was detectable using anti-GFP immunocytochemistry. Undetectable direct GFP fluorescence may be caused by levels of GFP expression that are below threshold, by protein misfolding, or by partial translation of the peptide containing the recognized epitope.

In most embryos, the onset of expression using microinjected DNA-bound sperm occurred after the maternal-embryonic transition, thought to occur in monkeys at the 4- to 8-cell stages, and can be mosaic and spatially restricted to as few as single blastomeres in a morula. Furthermore, dynamic low-light level imaging of the rhodamine-labeled DNA on the microinjected sperm demonstrated that DNA remains associated with the injected sperm within the oocyte cytoplasm (FIGS. 2A-2C).

A significant amount of plasmid DNA was observed on the sperm cell surface inside an oocyte fertilized by ICSI (FIGS. 1D and 1E). Rhesus embryos expressing green fluorescence protein by injecting DNA-labeled spermatozoa into mature oocytes were produced at an efficiency rate of 39.4% (FIGS. 1G and 1H). Rhesus pregnancies are routinely successful only when 4- to 8-cell embryos are transferred (Hewitson et al., 5 NAT. MED. 431-3, 1999) and thus, the growth of GFP-expressing blastocysts is an important experimental achievement.

Three rhesus pregnancies, with GFP-expressing embryos transferred at the 4- to 8-cell stages, resulted from seven embryo transfers. A healthy male was born at term, a set of anatomically normal twins (a male and a female) was stillborn at 35 days premature.

Fertilization by ICSI bypasses the normal plasma membrane interactions, which have been shown to exclude foreign genes adhering to the sperm. However, there is the possibility that the delivery of genetic material into an oocyte during ICSI may provide an alternative entryway for pathogens and consequent infection of the embryo. This may pose potential ramifications for colony management of endangered species and biomedical research animals.

Since ICSI circumvents the natural route of fertilization and the natural defense mechanism of an oocyte, several strategies are proposed to reduce or eliminate the potential pathogens adhering to the exterior of sperm chosen for ICSI. Ideally, these sanitizing treatments should employ both physical removal and chemical decontamination to ensure that only germ-free sperm are introduced. These chemical treatments preferably do not influence normal reproduction and therefore are preferably removed or neutralized prior to or during ICSI. The elimination of bacterial and viral pathogens could be accomplished by enzymatic hygienic treatments, particularly if the enzymes are physically bound so that they are removed prior to ICSI, or have a pH or other ion sensitivity such that they are neutralized within the cytoplasm. For example, proteinases and/or nucleases (DNases, RNases) could be used. Because even substantial washes are unlikely to result in complete decontamination, physical binding to an exogenous substrate is proposed. Polystyrene or magnetic beads, with brilliantly fluorescing dyes, are commercially available for enzyme cross-linkage.

A noninvasive assay for selecting among the myriad of potentially viable sperm (i.e., Berkovitz et al., 1 ANDROLOGIA 1-8, 1999) is important for transgenic methods. The binding of decontaminating enzymes to the zona pellucida may be a simple and feasible approach to the selection of sperm inside the perivitelline space after penetration through the zona pellucida, since this relies on a noninvasive and natural method for choosing the sperm for ICSI. In addition, this approach may also physically eliminate the exogenous material bound on the sperm since it is the first barrier during fertilization, and the conjugated enzymes might well destroy foreign infectious particles without interfering with the viability of the sperm for reproduction, since sperm retains its intact plasma membrane.

In the absence of any mature sperm, an alternative approach to ICSI involves the injection of spermatids into oocytes. In one study, the electrofusion of oocytes with round spermatids, the youngest male germ cells to have a set of haploid chromosomes, resulted in the birth of normal fertile mice (Ogura et al., 91 PROC. NATL. ACAD. SCI. USA 7460-62, 1994). Reports in humans demonstrate the possibility of round spermatid injection (ROSI) to produce viable embryos (Tesarik et al., 333 N. ENGL. J. MED. 525, 1995). While round spermatid injections have not led to the production of developmentally competent rhesus embryos, elongated spermatid injection (ELSI) has been successful

In a further aspect of the present invention, a transgenic reporter may be utilized to evaluate the gene delivery system and to select transgenic embryos. The use of a transgenic reporter is a powerful tool for determining successful delivery of exogenous DNA into a target cell. Many transgenic reporters are available but the most commonly and widely used is green fluorescent protein (GFP) which has been used in many applications including developmental and basic biological studies (Naylor, 58 BIOCHEM. PHARMACOL. 749-57, 1999; Ikawa et al., 430 FEBS LETT. 83-87, 1998; Rizzuto et al., 6 CURR. BIOL. 183-88, 1996).

Other trangenic reporters include, but are not limited to β-galactosidase, luciferase, and secreted placental alkaline phosphatase. The enzyme, β-galactosidase, catalyzes the hydrolysis of molecules containing β-gal linkages and the reaction product can be detected by a colormetric assay (Kubisch et al., 104 J. REPROD. FERTIL. 133-39, 1995; Chan et al., 52 MOL. REPROD. DEV. 406-13, 1999). Luciferase catalyzes the oxidative decarboxylation of luciferin producing a yellow-green light and its activity may be detected by photon imaging (Thompson et al., 92 PROC. NATL. ACAD. SCI. USA 1317-21,1995; Menck et al., 7 TRANSGENIC RES. 331-41, 1998). Secreted placental alkaline phosphatase (SEAP), a truncated form of placental alkaline phosphatase, is constitutively secreted and can be detected by chemiluminescence (Chan et al., 52 BIOL. REPROD. 137, 1995).

The expression of a transgene reporter may be used to monitor the development of a particular cell or tissue type. For example, a tissue or cell-specific promoter may be utilized to regulate the expression of the reporter. Using noninvasive imaging such as magnetic resonance imaging (MRI), positron emission topography (PET), or biophotonic imaging, the origin, migration, and fate of a particular cell may be analyzed. Thus, this technology may be used to monitor, for example, the growth of insulin-producing cells or neuronal cells (e.g., cells related to Parkinson's disease, Alzheimer's disease, and autism) during embryonic development.

Detection of transgenes in biopsied embryo samples by the highly sensitive method, polymerase chain reaction (PCR), has been widely used for selection of positive embryos for transfer (King and Wall, 1 MOL. REPROD. DEV. 57-62, 1988; Ninomiya et al., 1 MOL. REPROD. DEV. 242-48, 1989; Cousens et al., 39 MOL. REPROD. DEV. 384-91, 1994; Krisher et al., 78 J. Dairy Sci. 1282-88, 1994; Bowen et al., 50 BIOL. REPROD. 664-668, 1994). A relatively high false positive rate of fetuses and offspring may indicate the inaccuracy of the screening procedure (Burdon and Wall, 33 MOL. REPROD. DEV. 436-42, 1992; Cousens et al., 1994). A transgenic reporter protein is an alternative way to demonstrate the presence of the exogenous DNA after gene transfer into an embryo. Although transgene expression in early embryonic stages does not necessarily indicate the integration of exogenous DNA into the embryonic genome, the success in selecting GFP embryos and the creation of GFP-transgenic mice indicate the importance of transgenic reporters in embryo selection (Takada et al., 49 NAT. BIOTECH. 346-53, 1997).

The present invention also provides for a transgene under the control of regulatory elements, such as a promoter. A controllable promoter system or gene expression system is the most desirable. The choice of stage specific and/or a tissue specific promoter depends on the gene or target organ of interest. For a gene delivery system, the strong viral promoter, cytomegalovirus (CMV), is a suitable promoter as well as the protamine-1 promoter (O'Gorman et al., 94 PROC. NATL. ACAD. SCI. USA 14602-07, 1997). This promoter has been widely used in transgenic studies. Although it lacks specificity, its constitutive expression pattern will be an advantage during evaluation of gene delivery efficiency.

Other useful promoters for gene expression regulation include, but are not limited to, promoters for genes derived from viruses (e.g., Moloney leukemia virus), and promoters for genes derived from various mammals (e.g., humans, rabbits, dogs, cats, guinea pigs, hamsters, rats, and mice). Preferred promoters are those from the structural gene of interest (e.g., genes for insulin, erythropoietin, or platelet-derived growth factor). In another preferred embodiment, inducible promoters (e.g., tetracycline regulation system and metallothionein promoter) may be utilized to regulate the expression of the transgene (Iida et al., 70 J. VIROL. 6054-59, 1996; Palmiter, 91 PROC. NATL. ACAD. SCI. USA 1219-23, 1994).

Rhodamine-conjugation to DNA permits live imaging of the DNA dynamics. Confocal and conventional digital imaging verifies the binding of the DNA to the sperm, as well as the fate of the exogenous DNA after the sperm enters the egg cytoplasm. Rhodamine is an excellent fluorescent DNA marker for several reasons including its excitation by long wavelength (therefore less damaging lower energy) red light, and the avoidance of any confusion between the rhodamine DNA fluorescence and the green fluorescence from GFP transgene expression.

The dynamic imaging of the expression of GFP may be examined by fluorescent microscopy using FITC filters. In the methods of the present invention, GFP expression may be followed from the 2-cell to blastocyst stages. Expression is dependent on the transcriptional activity of the embryo. Two different types of expression can be expected. Transgene expression can be derived from an integrated transgene or from a non-integrated exogenous DNA (transient expression). In case of a successful integration, expression is expected following the maternal to embryonic transition in transcription. Transient expression can be expected at anytime during in vitro culture when active transcription machinery is present. Therefore, expression of exogenous DNA is a good reference for successful gene delivery but successful integration must be confirmed by the production of transgenic offspring or by analysis of successful integration of exogenous DNA into the embryonic genome by in situ PCR.

Determination of the viability parameters of oocytes and embryos imaged either by conventional or confocal microscopy is critical for the later stages of selecting GFP-expressing embryos or blastomeres for embryo transfer. Thus, the light intensities and exposure durations that will prevent normal development in control zygotes and embryos may be determined by quantitating exposure with viability. With this data, low light level imaging may be optimized so that fluorescence images will be collected using light intensities of only a small percentage of the amount that may compromise later development.

The expression of GFP, PCR analysis, Southern blot analysis, fluorescence in situ hybridization (FISH), and in situ PCR may be utilized to examine the presence of a transgene in tissue samples and to identify the integration of the exogenous DNA into the target cell genome. Southern blot analysis demonstrates the size of the provirus, integration pattern, and possible rearrangement of the transgene. In situ PCR identifies the chromosomal location of the transgene. The interpretation of the analyses varies among samples and depends on the time when tissue samples are collected.

Three developmentally progressive stages may be analyzed for the presence of a transgene: blastomeres, fetuses, and offspring. In blastomeres, traditional PCR analysis cannot distinguish between the non-integrated free-form exogenous DNA and the integrated transgene. In order to determine successful integration in blastomeres, either FISH or in situ PCR may required. Both methods can exactly define the location of the transgene in the target cell genome. The advantage of in situ PCR is the amplification of the signal, which can then be detected with FISH. In the case of successful integration, the localization of the FISH signal corresponds to the location of nuclear DNA.

PCR analysis becomes more reliable at the fetal stage because the non-integrated free-form exogenous DNA has been degraded. In offspring, PCR is a reliable screening method for transgenesis because non-integrated free-form exogenous DNA does not exist and integration can be further confirmed by Southern blot analysis. The ultimate success of transgenesis will be asserted by in situ PCR and Southern blotting.

The success of transgenesis may be ascertained by direct low-light level GFP fluorescence on live embryos during preimplantation period. To determine if GFP expression occurs at levels below detection limits, or if incorrect folding or mis-expression might have occurred, monoclonal antibodies to GFP may be employed to examine individual blastomeres by indirect immunocytochemistry using a fluorophore that does not preclude direct GFP fluorescence. Single cell (i.e., blastomere) PCR may be used to determine the presence of the GFP transgene, and, if the signal is lost, the frequency and timeframe of its destruction. Finally, the normalcy of development may be evaluated using available cell cycle checkpoint markers (i.e., DNA replication, mitosis, and cytokinesis), as well as markers of intracellular architecture (i.e., cytoskeletal and endomembrane probes).

Another embodiment of the present invention relates to the production of transgenic animals by chimeric construction. A chimera is a mosaic organism composed of cells of different genetic origin. Generally, the blastomeres of several embryos are completely disassociated followed by reaggregation of blastomeres from different embryos and then development to the blastocyst stage. Aggregation chimeras have been produced successfully, not only within a species (Gardner, 6 ADV. BIOSCI. 279-301, 1971; Stevens, 276 NATURE 266-67, 1978; Stern and Wilson, 28 J. EMBRYOL. EXP. MORPHOL. 247-54, 1972), but also between them (Fehilly et al., 1984) and have resulted in live offspring. When constructing chimeras, same sex blastomeres must be used to avoid possible developmental abnormalities. Although viable, mixed sex chimeras inevitably result in androgenization of the offspring (Patek et al., 1991). A single XY-containing blastomere can cause androgenization and testis determination in otherwise XX embryos (Koopman et al., 1990) and change the expected phenotype. The presence of XX blastomeres in a predominantly XY embryo impairs testis formation and consequently fertility. When creating chimeras, mixing of blastomeres from sibling embryos inevitably results in genetic mosaicism. Different blastomeres can contain transgenes at different insertion sites. For example, if the liver was derived from a single blastomere, all the liver cells should display the same localization of the transgene (by FISH or in situ PCR). On the other hand, if the liver developed from two or more blastomeres from sibling embryos, it can be expected that the transgene will localize to different chromosomes or chromosomal sites in different liver cells. Because plasmid integration and gene expression is random using sperm-mediated transfer, the level of “transgenesis” in embryos may be increased by creating chimeras.

Transgenic offspring may be detected by any of several means well known to those skilled in the art. Non-limiting examples include Southern blot or Northern blot analyses, using a probe that is complementary to at least a portion of the transgene. Western blot analysis using an antibody against the protein encoded by the transgene may be employed as an alternative or additional method for screening for the presence of the transgene product. A DNA sample may be prepared from a tissue or cell and analyzed by PCR for expression of the transgene.

Alternative or additional methods for evaluating the presence of the transgene include, without limitation, biochemical assays such as enzyme and/or immunological assays, histological stains for particular marker or enzyme activities, flow cytometric analysis, in situ hybridization of mRNA analysis, and FACS analysis of protein expression. Analysis of the blood may also be useful to detect the presence of the transgene product in the blood, as well as to evaluate the effect of the transgene on the levels of various types of blood cells and other blood constituents.

Animal tissue may also be analyzed directly, for example, by preparing tissue sections. In some embodiments, it may be preferable to fix the tissue (e.g., with paraformaldehyde or formalin). Tissue sections may be prepared frozen, or may be paraffin-embedded. Slides of animal tissue may be used for immunohistochemistry, in vitro hybridization, or histology (e.g., hematoxylin and eosin staining).

Transgenic cells, genetically identical cells, and stem cells derived from primates are invaluable for the study of numerous diseases (e.g., aging, AIDS, cancer, Alzheimer's disease, autoimmune diseases, metabolic disorders, obesity, organogenesis, psychiatric illnesses, and reproduction). Furthermore, the importance of these cells for molecular medicine and the development of innovative strategies for gene therapy protocols should not be minimized. For example, clinical strategies may include cloning, assisted reproductive technologies, transgenesis, and use of totipotent and immortalized embryonic germ (EG) and stem cells (ES). In addition, identical, transgenic and/or immortalized, totipotent EG or ES-derived cells may be ideal preclinical models in identifying the molecular events related to infertility, gametogenesis, contraception, assisted reproduction, the genetic basis of infertility, male versus female meiotic cell cycle regulation, reproductive aging, and the non-endocrine basis of idiopathic infertility.

These transgenic technologies may also be utilized to study human development, particularly pre- and post-implantation development, body axis specification, somitogenesis, organogenesis, imprinting, extra-embryonic membrane allocation, and pluripotency. Using dynamic noninvasive imaging of transgenic reporters, the cell allocation in the primate fetus may be identified throughout pregnancy and life. Cloning and transgenesis may also be used to discover disease mechanisms and to create and optimize molecular medical cures. For example, monkeys created with a genetic knockout for a specific gene may accelerate discovery of the cures for cancer, arteriosclerosis causing heart disease and strokes, inborn errors of metabolism and other fetal and neonatal diseases, Parkinson's disease, polycystic kidney disease, blindness, deafness, sensory disorders, storage diseases (Lesch-Nyan and Zellwegers) and cystic fibrosis. These transgenic animals may also be amenable for evaluating and improving cell therapies including diabetes, liver damage, kidney disease, artificial organ development, wound healing, damage from heart attacks, brain damage following strokes, spinal cord injuries, memory loss, Alzheimer's disease and other dementia, muscle and nerve damage.

Thus, the present invention also relates to methods of using transgenic embryonic cells to treat human diseases. Specifically, the methods to produce transgenic animals and transgenic primates, described in the present invention, may also be used to create transgenic embryonic stem cells.

Briefly, following fertilization, an egg divides over a period of days to form a blastocyst which, generally, is a hollow ball of cells having an inner cell mass and a fluid-filled cavity, both encapsulated by a layer of trophoblast cells. Cells from the inner cell mass of an embryo (i.e., blastocyst) may be used to derive a cell line referred to as embryonic stem (ES) cells, and these cells may be maintained in tissue culture (see e.g., Schuldiner et al., 97 PROC. NATL. ACAD. SCI. USA 11307-12, 2000; Amit et al., 15 DEV. BIOL. 271-78, 2000; U.S. Pat. No. 5,843,789; U.S. Pat. No. 5,874,301). In general, stems cells are relatively undifferentiated, but may give rise to differentiated, functional cells. For example, hemopoietic stem cells may give rise to terminally differentiated blood cells such as erythrocytes and leukocytes.

Using the methods described in the present invention (e.g., TransgenICSI, retroviral gene transfer), transgenic primate embryonic stem cells may be produced which express a gene related to a particular disease. For example, transgenic primate embryonic cells may be engineered to express tyrosine hydroxylase which is an enzyme involved in the biosynthetic pathway of dopamine. In Parkinson's disease, this neurotransmitter is depleted in the basal ganglia region of the brain. Thus, transgenic primate embryonic cells expressing tyrosine hydroxylase may be grafted into the region of the basal ganglia of a patient suffering from Parkinson's disease and potentially restore the neural levels of dopamine (see e.g., Bankiewicz et al., 144 EXP. NEUROL. 147-56, 1997). The methods described in the present invention, therefore, may be used to treat numerous human diseases (see e.g., Rathjen et al., 10 REPROD. FERTIL. DEV. 31-47, 1998; Guan et al., 16 ALTEX 135-41, 1999; Rovira et al., 96 BLOOD 4111-117, 2000; Muller et al., 14 FASEB J. 2540-48, 2000).

To develop a model for a specific human disease, a transgenic monkey may be produced by the following steps: 1) production of a transgenic monkey displaying gene line transmission; 2) production of monkey offspring clones; 3) establishment of pluripotent cell lines and creation of chimeric primates; 4) development of noninvasive procedures to monitor pregnancy, transgenesis efficiency, and fetal and offspring outcomes; 5) development of homologous recombination to generate knockouts for specific genes; 6) creation of identical primates for a devastating human disease (e.g., Her-2 or BRCA-½ knockout modeling breast and ovarian cancer); 7) development of gamete, gonad, and embryo storage procedures that both retain full reproduction potential and permit inexpensive archival storage; 8) development of procedures for propagating uninfected primates both on- and off-site.

### EXAMPLES

The present invention is further illustrated by the following examples which should not be construed as limiting in any way. The contents of all cited references (including literature references, issued patents, published patent applications, and co-pending patent applications) cited throughout this application are hereby expressly incorporated by reference.

### Example 1

### TransgenICSI Procedures

One of the advantages for delivery of DNA bound to the surface of spermatozoa is that exogenous DNA can be of any size. Linear DNA construct has higher gene integration efficiency after pronuclear injection (Brinster et al., 82 PROC. NATL. ACAD. SCI. USA 4438-42, 1985). Treatment of decondensed sperm nuclear chromatin with a unique restriction enzyme that linearizes exogenous DNA creates compatible cutting sites. Exogenous DNA integration is believed to be a random event and depends on DNA breakage. Creation of compatible cutting sites enhance integration events by providing a partial non-random integration site, compatible to the linearized exogenous DNA. Evaluation of sperm after DNA incorporation is performed by PCR and in situ PCR. The PCR technique may not be an adequate method because it does not distinguish between free and incorporated DNA. Thus, in situ PCR is an alternative, which can demonstrate the location of the transgene in the chromatin. Residual plasmid DNA is rinsed from the oocyte surface and the rhodamine signal is monitored by confocal microscopy prior to extraction of nuclear DNA for PCR analysis. To confirm the presence of exogenous DNA in each blastomere, individual blastomeres are isolated and analyzed by PCR.

Sperm collection and preparation. Rhesus monkey semen was used for plasmid DNA labeling. Rhesus males of proven fertility have been trained to routinely produce acceptable semen samples by penile electroejaculation (Bavister et al., 28 BIOL. REPROD. 983-99, 1983). After liquefaction of the coagulated ejaculate, the liquid semen was washed in 5 ml of TALP-HEPES by centrifugation at 400×g for 5 minutes. After resuspension of the pellet in 1 ml TALP-HEPES, a small sample was removed for structural analysis, while the remainder was counted and diluted to a concentration of 20×10⁶ sperm/ml in equilibrated TALP (1 ml) in a 15 ml conical tube.

Plasmid construction. The plasmid DNA, which included a green fluorescent protein (GFP) cDNA and a rhodamine binding site, was constructed under the control of cytomegaloviris (CMV) promoter (Gene Therapy System, San Diego, Calif.). Rhodamine labeling of the plasmid DNA was performed as described by manufacturer.

GFP cDNA, under the control of CMV promoter, was employed. The CMV promoter was selected since it is a strong viral promoter widely used in transgenic studies. Although it lacks specificity, its constitutive expression pattern is an advantage during evaluation of gene delivery efficiency. The use of the GFP transgenic reporter is a powerful tool for determining successful delivery of exogenous DNA into oocytes and embryo. Although fluorescent microscopy is required, successful production of transgenic mice after GFP selection suggests limited or no effect on embryo and fetal development.

The use of a fluorescent DNA marker, rhodamine-conjugation to DNA, permits live imaging of the DNA dynamics during IVF as well as ICSI. Confocal and conventional digital imaging verifies the binding of the DNA to the sperm, as well as the fate of the exogenous DNA after the sperm enters the egg cytoplasm. Rhodamine was chosen for several reasons including its excitation by long wavelength (therefore less damaging lower energy) red light, and avoidance of any confusion between the rhodamine DNA fluorescence and the anticipated green fluorescence from GFP transgene expression.

Sperm nuclei preparation and DNA association. To enhance internalization of exogenous DNA into the sperm nucleus and integration into the sperm genome, rhesus sperm nuclei were subjected to in vitro decondensation and treated with restriction enzymes to create nicks on both the exogenous DNA and the sperm genome. The restriction enzyme was used to linearize the DNA construct and cut decondensed sperm nuclear chromatin to create compatible cutting sites.

A motile fraction of rhesus sperm was isolated by a 10-minute spin at 700×g on a 45:90% Percoll density gradient. The pellet was resuspended in 5 μg/ml lysolethicin in KMT medium (100 mM KCl, 2 MM MgCl₂,10 mM Tris-HCl (pH 7.0), and 5 mM EGTA) at 20° C. for 10 minutes, followed by a 10-minute rinse in 3% BSA in KMT. Sperm were then treated with 5 mM DTT (pH 8.2) in KMT at 37° C. for 1 hour followed by three washes in KMT. After washing, the DTT-treated spermatozoa were incubated with the DNA plasmid. The labeled sperm was diluted 1:10 in thawed extract (approximately 1000 sperm/μl) containing the DNA restriction enzyme, Pvu I. The extract:sperm mixture was incubated for 1 hour at 37° C.

The decondensed sperm nuclei (˜9-10 μm in diameter) were isolated by diluting the extract 1:10 in Pipes buffer (80 mM Pipes (pH 6.8), 5 mM EGTA, 1 mM MgCl₂) and placing about 10 μl under oil adjacent to the oocytes. ICSI was then performed, but with a slightly larger diameter ICSI needle to accommodate the increased size of the sperm nucleus. Parthenogenic development, where the injected sperm triggers oocyte activation, and maybe even contributes the sperm centrosome but not the paternal genome were monitored, as are the sex ratio of the embryos, i.e., the frequency of male embryos.

Binding of exogenous DNA to sperm. Sperm was labeled with plasmid DNA encoding GFP cDNA conjugated with rhodamine (Gene Therapy System, San Diego, Calif.). The sperm (1×10⁶/14 μl) was mixed with 500 ng (1 μl) of plasmid DNA (Rh-CMV-GFP) and incubated at 37° C. for 30 minutes. Labeled sperm were washed and centrifuged three times in TALP-HEPES buffer, followed by fluorescent microscopic imaging before subsequent use in intracytoplasmic sperm injection. Trypsin-treatment of DNA-bound spermatozoa was performed by incubation with 2 mg/ml of trypsin (Sigma, St. Louis) in phosphate buffered saline for 30 minutes prior to washing. Rhodamine-tagged plasmid DNA bound avidly to sperm and served as a dynamic fluorescent marker (FIGS. 1A-1C).

Cell-free sperm nuclear decondensation using Xenopus egg extracts. To enhance plasmid binding efficiency, sperm nuclei were exposed to Xenopus cell-free extract. The Xenopus cell-free extract were prepared according to Murray, (36 METH. CELL BIOL. 581-605, 1991). Xenopus oocytes were induced to mature by injection of 100 I.U. PMSG into the dorsal lymph sac of X. laevis on day one. A second injection of 500 I.U. hCG, on day four, induced the females to lay their eggs. Eggs, which had been laid into MMR medium, were collected 10-12 hours post-hCG injection. Debris was removed by rinsing in MMR, and the eggs were dejellied by a 6-minute exposure to cysteine dejellying solution (100 mM KCl, 0.1 mM CaCl₂, 1 mM MgCl₂, and 2% w/v L-cysteine, pH 7.8). Following two rinses in 0.2×MMR, the eggs were rinsed 4 times in XB (Extract Buffer: 100 mM KCl, 0.1 mM CaCl₂, 1 mM MgCl₂, 10 mM HEPES (pH 7.8), 50 mM sucrose, and 5 mM EGTA), and then twice more in XB containing the protease inhibitors leupeptin, chymostatin, and pepstatin A (at 10 μg/ml each). The eggs were transferred with a minimal volume of XB containing protease inhibitors and 100 μg/ml cytochalasin B (to prevent gelation) to centrifuge tubes. The eggs were packed during a two-minute centrifugation at 2000 rpm in a Beckman SW28 rotor; the excess buffer and Versalube were removed. The eggs were then subjected to a stratifying centrifugation step for 20 minutes in a SW28 rotor at 20,000 rpm. The cytoplasmic layer was removed by puncturing the side of the UltraClear™ centrifuge tube. The cytoplasmic extract was then fortified with an “Energy Mix” (150 mM creatine phosphate, 20 mM ATP (pH 7.4), 2 mM EGTA (pH 7.7), and 20 mM MgCl₂: 5 μl/100 μl extract) containing cytochalasin B and protease inhibitors. For freezing, sucrose was added to the extract at a final concentration of 200 mM, and the aliquots were flash-frozen in liquid nitrogen and stored at −70° C.

Rhesus follicle stimulation. Hyperstimulation of female rhesus monkeys exhibiting regular menstrual cycles was induced with exogenous gonadotropins (Zelinski-Wooten et al., 51 HUM. REPROD. 433-40, 1995; Meng et al., 57 BIOL. REPROD. 454-459, 1997; Hewitson et al., 13 HUM. REPROD. 3449-55, 1998). Beginning,at menses, females were down-regulated with GnRH antagonist (Antide; Ares Serono, Aubonne, Switzerland; 0.5 mg/kg body weight, s.c.) for 6 days during which recombinant human FSH (r-hFSH; Organon Inc., West Orange, N.J.; 30 IU, i.m.) was administered twice daily, followed by 1, 2, or 3 days of r-hFSH+r-hLH (r-hLH; Ares Serono; 30 IU each, i.m., twice daily). Ultrasonography was performed on day seven to confirm adequate follicular response. r-hCG (Serono, Randolph, Mass.; 1000 IU) is administered for ovulation when follicles were 3-4 mm.

Rhesus follicular aspiration by laparoscopy. Follicular aspiration was performed 27 hours post-hCG administration. Oocytes were aspirated from follicles using a needle suction device lined with Teflon tubing (Renou et al., 35 FERTIL. STERIL. 409-12, 1981, and modified by Bavister et al., 1983). Multiple individual follicles were aspirated with continuous vacuum at approximately 40-60 mmHg pressure into heparinized blood collection tubes. Collection tubes were immediately transported to a dedicated primate oocyte/zygote laboratory for oocyte recovery and evaluation of the maturation stage.

Collection and evaluation of Rhesus oocytes. The contents of each collection tube were diluted in TALP-HEPES supplemented with 2 mg/ml hyaluronidase. Oocytes were rinsed and then transferred to pre-equilibrated CMRL medium containing 3 mg/ml BSA (CMRL-BSA) and supplemented with 10 μg/ml porcine FSH and 10 IU/ml hCG, prior to evaluation of maturational state. Metaphase II-arrested oocytes, exhibiting expanded cumulus cells, a distinct perivitelline space, and first polar body, were maintained in CMRL-BSA for up to 8 hours before fertilization. Immature oocytes were matured in CMRL-BSA plus hormones for up to 24 hours (Bavister et al., 1983; BOATMAN, IN VITRO GROWTH OF NON-HUMAN PRIMATE PRE- AND PERI-IMPLANTATION EMBRYOS 273-308 (B. D. Bavister, ed., Plenum Press 1987)).

Intracytoplasmic sperm injection. Holding pipettes (O.D. 100 μm; I.D. 20 μm) and microinjection needles (O.D. 6-7 μm and I.D. 4-5 μm) with a 50° bevel and a short, sharp, point (Humagen, Inc., Charlottesville, Va.) were mounted on a Nikon Diaphot microscope equipped with Hoffman modulation contrast (HMC) optics. The holding pipette was held in a Narishigi (MN-151) manipulator attached to a Hamilton syringe. The injection pipette was mounted in a motorized Eppendorf (5170) micromanipulator attached to a Narishigi (IM-6) injection system. Injections were carried out at 32° C. in 100 μl of TALP-HEPES placed in the lid of 100 mm tissue culture dish and covered with light mineral oil (Hewitson et al., 55 BIOL. REPROD. 271-80, 1996; Hewitson et al., 1998). Capacitated, hyperactivated sperm were diluted 1:10 in 10% polyvinylpyrrolidone (PVP). A single sperm was aspirated tail-first from the sperm-PVP drop into the microinjection needle and transferred to the oocyte-containing drop. Oocytes were immobilized with the polar body at 12 o'clock, and the injection needle was inserted through the zona into the cytoplasm. The oolemma was breached by gentle cytoplasmic aspiration when the sperm is released back into the oocyte. Microinjected oocytes were examined with 40× HMC objective to verify the presence of a single sperm within the cytoplasm.

Dynamic imaging of DNA-bound sperm during ICSI. Images were captured on an inverted TE-300 Nikon microscope equipped with a Princeton CCD camera and Metamorph software (Universal Imaging, West Chester, Pa.). Final images were prepared using Adobe Photoshop (Adobe Systems Inc., MotntainView, Calif.). Rhodamine-tagged DNA remained on the surface of the microinjected sperm following ICSI (FIGS. 1D and 1E).

Culture of TransgenICSI oocytes and embryos. Oocytes were washed in equilibrated TALP and returned to culture in 100 μl TALP under oil. Fertilization was assessed within 3-6 hours by detection of the second polar body using HMC optics. The number of pronuclei was assessed between 12-16 hours post-injection. After completion of the first cleavage division (24-28 hours post-injection), 2-cell embryos were co-cultured in CMRL+10% FCS (Hyclone Laboratories, Inc., Logan, Utah) on Buffalo rat liver cell monolayers (BRL 1442; ATCC, Rockville, Md.) seeded in 100 μl overlaid with oil. Embryos were selected at the 3- to 16-cell stage for transfer into staged recipients.

Embryo transfer. Rhesus females with normal menstrual cycles synchronous with the egg donors were screened as potential embryo recipients. Screening was performed by collecting daily blood samples beginning on day 8 of the menstrual cycle (with first day of menses as day 1) and analyzed for serum progesterone and estrogen. Timing of ovulation was detected by a significant decrease in serum estrogen and an increase in serum progesterone to above 1 ng/ml. Surgical embryo transfers were performed on day 2 or 3 into the oviduct of the recipient, by mid-ventral laparotomy. The oviduct was cannulated and two 4- to 8-cell stage embryos were transferred via a small catheter.

To confirm implantation, blood samples were collected daily and analyzed for serum estrogen and progesterone (Lanzendorf et al., 42 BIOL. REPROD. 703-11, 1990) and pregnancies confirmed by ultrasonography on day 35 post-transfer. During ultrasound, measurements were taken of total fetal length, femur length, head circumference, fetal cardiac activity, and size of yolk sac. Ultrasound was performed once more, during the second trimester, to determine developmental normalcy. In recipients that maintained adequate estrogen and progesterone levels, but who were deemed not pregnant by ultrasound examination, blood samples were analyzed for serum monkey chorionic gonadotropin (mCG) measured by an LH bioassay (Ellinwood and Resko, 22 BIOL. REPROD. 955-63, 1980).

Births and infants. Since rhesus monkeys occasionally experience pre-eclampsia and newborns can be lost due to complications at birth, babies were delivered by cesarean section at approximately 155 days of pregnancy. Babies were weighed, measured, and their head circumference recorded, and then kept in an incubator until the mothers recovered from surgery. The babies were daubed in placental blood prior to re-introducing them to their mothers so the possibility of rejection was minimized. Some of the placental tissue was collected and examined for transgenesis. Babies remained with their mothers until weaning at six months of age. Tissues from offspring were collected by biopsy and were examined for transgenesis.

Detection of embryonic GFP-transgene expression by live, digital lowlight level epifluorescence imaging. Live embryos were imaged with a Nikon TE-300 inverted microscope equipped with FITC filters and a Princeton CCD camera. Images were captured and analyzed by Metamorph software (Universal Imaging, West Chester, Pa.).

Detection of embryonic GFP-transgene expression by immunocytochemistry. To detect GFP expression, selected embryos were fixed and immunostained with a polyclonal rabbit anti-GFP antibody (ClonTech, CA). After zona pellucida removal with 0.5% pronase, embryos were attached to polylysine-coated coverslips and fixed for 1 hour in 2% formaldehyde in TALP-HEPES. Fixed embryos were permeabilized in 0.1 M PBS containing 2% Triton X-100 detergent for 40 minutes, followed by incubation for 30 minutes in a PBS blocking solution containing 150 mM glycine and 3 mg/ml BSA. The primary GFP antibody was diluted 1:100 in PBS and applied for 1 hour at 37° C. After a 30-minute wash in PBS with 0.1% Triton detergent, GFP primary antibody was detected using rhodamine-conjugated anti-rabbit IgG secondary antibody. DNA was labeled with 5 μg/ml Hoechst 33342 added to the penultimate rinse and embryos. The samples were then mounted in Vectashield antifade (Vector Labs, CA) and examined with a Zeiss Axiphot epifluorescent microscope equipped with appropriate filters and high numerical aperture objectives.

Detection of transgene in Rhesus tissue by PCR. Tissues from the two stillborns were collected and maintained at −20° C. until DNA extraction. Buffy-coats collected from blood were isolated by centrifugation using PMN isolation medium (Robbins Scientific Corporation). DNA was extracted by proteinase K digestion followed by phenol-chloroform extraction. After ethanol precipitation, the DNA pellet was resuspended in Tris-EDTA buffer (pH 8.0). Genomic DNA (1 mg) was used for PCR analysis using GFP specific primer set (GFP#1: TGAACCGCATCGAGCTGAAG, and GFP#2: CGATGTTGTGGCGGATCTTG). The DNA mix contained 200 mM dNTP (Pharmacia), 1.0 mM of each primer, 1.5 mM of MgCl₂, 0.1 volume of 10× reaction buffer, and 1 unit of Taq DNA polymerase (Promega, Madison, Wis.). The amplification cycle was 94° C. for 5 minutes followed by thirty cycles of 94° C. for 2 minutes, 60° C. for 2 minutes, and 72° C. for 2 minutes. PCR products were separated on a 2% agarose gel.

Detection of DNA replication. DNA synthesis was determined using Bromodeoxyuridine (BrdU; Boehringer Mannheim Corp., IN) after fixation, or after microinjection of Oregon Green dUTP (Molecular Probes, OR) in a living oocyte or embryo. Within the first hour after TransgenICSI, oocytes were transferred to TALP containing either 50 μM BrdU or were microinjected with 1 μM Oregon Green dUTP. After in vitro culture at 37° C. to an appropriate stage, embryos were either permeabilized and fixed for 20 minutes at −20° C. (70% ethanol in 50 mM glycine buffer, pH 2.0), or were mounted as living embryos on slides for examination by epifluorescence or confocal microscopy. For fixed embryos, BrdU was labeled with a mouse IgG monoclonal antibody (6 μg/ml) to BrdU (Boehringer), and detected with a 1:50 dilution of fluorescein-conjugated goat anti-mouse IgG secondary antibody. DNA was labeled with 5 μg/ml Hoechst 33342 in the penultimate PBS rinse and the slides were observed for the incorporation of BrdU. DNA synthesis in the living embryos after microinjection with 1 μM Oregon Green dUTP was detected by conventional epifluorescence or confocal microscopy as described by Carroll et al. (206 DEV. BIOL. 232-47, 1999). Briefly, free Oregon Green dUTP nucleotides not incorporated into the DNA of a TransgenICSI embryo were reduced by photobleaching of a cytoplasmic site near the nucleus using high intensity fluorescent light (˜488 nm). After photobleaching, non-incorporated Orgeon Green dUTP is dimmer than the brightly fluorescent nuclei containing DNA-bound Oregon Green dUTP, indicative of DNA synthesis. Images of the nuclear fluorescence were captured by a chilled CCD camera or the photodetector on the confocal microscope.

Detection of mitosis. The zonae were removed from zygotes and embryos by a 2-7 minute incubation in 0.5% pronase prepared in TALP-HEPES. After a 30-minute recovery at 37° C., zona-free oocytes were attached to polylysine-coated coverslips and permeabilized in Buffer M (Simerly and Schatten, 225 METH. ENZYMOL. 516-52, 1993) containing 3% Triton X-100 detergent and 8% methanol for 10 minutes. Permeabilized zygotes were further fixed in cold (−10° C.) absolute methanol for 20 minutes before rehydration with 0.1 M PBS containing 0.1% Triton. Microtubule localization was performed using E-7 (1:5), a mouse monoclonal antibody to β-tubulin that has wide cross reactivity to β-tubulin from numerous species (Chu and Klymkowsky, 8 FIRST INTER. SYMP. CYTOSKEL. DEV. 140-42, 1987). E-7 antibody was detected using either rhodamine or Cy5-labelled goat anti-mouse IgG secondary antibody (Zymed Laboratories, Inc., San Francisco, Calif.). To detect expression of GFP, a commercially available rabbit polyclonal anti-GFP antibody was used according to the manufacturer's recommendation (1:100; Clontech, CA). The primary antibody was applied for 40 minutes at 37° C. before rinsing with PBS with 0.1% Triton. A goat anti-rabbit IgG secondary antibody conjugated to either rhodamine or Cy5 was used to detect anti-GFP primary antibody. DNA was fluorescently detected with 5 μg/ml Hoechst 33342 added to the penultimate rinse. Coverslips were mounted in Vectashield and examined using conventional immunofluorescence and laser-scanning confocal microscopy.

Embryos produced by TransgenICSI were examined by fluorescent microscopy at various times during culture. The GFP cDNA is controlled by a CMV promoter which is a strong viral promoter and believed to be constitutively expressed during embryonic development. In the event that embryos were PCR positive but GFP expression was not detected, RT-PCR was used to determine if the transcriptional machinery in the embryos was active.

Detection of GFP expression by RT-PCR. Following TransgenICSI, individual embryos were washed in PBS and transferred to 0.2 ml thin wall microcentrifuge tubes with 5 μl of DEPC-treated water. An oligo-d(T) primer was used in the reverse transcription reaction (RT) to produce a cDNA template for the next amplification step. The RT-PCR product was then amplified by PCR. In brief, a specific primer set for GFP was used, GFP#1: TGAACCGCATCGAGCTGAAG, and GFP#2: CGATGTTGTGGCGGATCTTG, which yields a 156 bp fragment. The PCR reaction was performed in a final volume of 50 μl. Approximately 1 μg of genomic DNA and 1 ng of plasmid DNA in 5 μl of Tris-EDTA buffer (pH 8.0) was used as the template. Forty-five microliters (45 μl) of the PCR reaction mix (200 μM dNTP, 1.0 μM of each primer, 1.5 mM MgCl₂, 0.1 volume 10× reaction buffer, and 1 U Taq polymerase) was added to each sample. The cycles were 94° C. for 2 minutes, 50° C. for 2 minutes and 72° C. for 2 minutes. After 30 cycles, the PCR products were separated by electrophoresis on a 2% agarose gel.

Conventional and confocal immunofluorescence. Embryos were examined using both conventional immunofluorescence and laser-scanning confocal microscopy. Conventional fluorescence microscopy was performed using a Zeiss Axiophot microscope with high numerical aperture objectives, since photobleaching was negligible. Data was collected using black and white Tri-X and color Ektachrome film and then digitally recorded using a cooled CCD camera (Princeton Instruments Inc., Trenton, N.J.). Laser-scanning confocal microscopy was then performed using a Leica TCS-NT equipped with Krypton-Argon/Helium-Neon laser for the simultaneous excitation of fluorescein, rhodamine, Cy-5, and UV. The confocal microscope provides an accurate image of the inner cell mass (ICM) and trophectoderm (TE) cells of blastocysts. Digital images were recorded and archived on Jazz disks. Digital data was downloaded to a dye-sublimation printer (Sony) using Adobe Photoshop (Adobe Systems Inc., MountainView, Calif.). Measurements and analysis were performed using Metamorph software (Universal Imaging, West Chester, Pa.) and NIH Image, an image analysis program.

Establishment of Rhesus embryonic stem cells. The zonae were removed from in vitro-produced GFP-infected 2-cell rhesus embryos with pronase. The zona-free embryos were washed twice. Each embryo was transferred to a 72-microwell plate containing mitomycin-treated mouse fetal fibroblasts (MFF) and 5 μl of CR1aa supplemented with 15% heat-treated fetal bovine serum. The medium was changed every day until the size of the colony was dense enough to transfer to a 35 mm dish plated with mitomycin-treated MFF. The medium was changed to DMEM supplemented with 15% FBS and 0.1 mM β-mercaptoethanol. The medium was then changed every one or two days and selected regions of the colony were cut with a sharp pipette and pasted onto a new inactivated MFF layer.

Establishment of primary cell cultures from fetuses. Primary cultures were established from fetal tissues originating from all three germ layers according to established cell culture protocols. Tissues were minced with scissors under sterile conditions, transferred to 0.25% trypsin-EDTA and incubated for 1 hour at 37° C. Dissociated cells were transferred to tissue culture flasks and grown in DMEM, supplemented with 10% FCS.

### Example 2

### DNA Integration in TransgenICSI

PCR analysis of tissues obtained from Rhesus fetuses and offspring. DNA was extracted from nucleated blood cells, the placenta at the time of delivery, and tissues derived from the three germ layers. The blood was collected in a heparinized tube and centrifuged at 2500×g for 15 minutes at 4° C. The buffy coat, containing the white blood cells, was transferred to a 15 ml conical tube. Skin tissue was minced using scissors and transferred to a 15 ml conical tube. Two volumes of hypotonic lysis buffer (150 mM NH₄Cl, 10 mM KHCO₃, 10 mM Na₂-EDTA) was added to lyse the red blood cells. After centrifuging for 30 seconds at 2500×g, the supernatant was discarded and the process was repeated until the red blood cells were removed. White blood cells or minced tissue were resuspended in 3 ml salt digestion buffer (10 mM Tris-HCl, 400 mM NaCl, 2% SDS, 50 mM EDTA, pH 8.0). Proteinase K (50 μl; 10 mg/ml) and trypsin (70 μl; 5 mg/ml) were added and the samples were vortexed before incubation at 50° C. with shaking (150 rpm) until the tissue dissolved (2-4 hours). Following digestion, RNase (100 U) was added and the samples were then incubated for 1 hour at 50° C. with shaking (150 rpm). One-third volume of 5 M NaCl was added and the samples were mixed by inverting the tube, and then centrifuged at 3,000×g for 30 minutes. Supernatant was removed and transferred to a tube containing 2 volumes of 95% ethanol. The DNA precipitate was then removed by a sterile pipette tip, transferred to a microcentrifuge tube, and washed twice with 70% ethanol. The DNA samples were analyzed by PCR.

Southern blot analysis. Genomic DNA (10 μg) was digested with restriction enzymes and the DNA fragments were separated by electrophoresis on a 0.8% agarose gel. The gel was then subjected to acid depurination (washing the gel in 0.25 N HCl for 15 minutes) and denaturation (washing the gel twice in 1.5 M NaCl, 0.5 M NaOH for 20 minutes) at room temperature. Following this procedure, the DNA fragments were transferred to Hybond-N+ nylon membranes (Amersham). The membrane was then neutralized by washing in a solution of 1M Tris·Cl (pH 8.0) and 1.5 M NaCl for 15 minutes. The membrane was baked for 1 hour at 80° C. in order to crosslink the DNA fragments to the membrane. The baked membrane was transferred to a hybridization tube and 6 ml of pre-heated Rapid Hybridization Buffer (Amersham) was added. The membrane was then incubated in a hybridization oven at 65° C. with rolling for 1 hour. A ³²P-labeled probe (1×10⁶ cpm/ml) was then added to the hybridization solution and the membrane was hybridized at 65° C. for another 40 to 60 minutes. Following hybridization, the membrane was washed four times at 65° C. with high stringency buffer, and exposed to X-ray film at −80° C. for 2 to 3 weeks. Digestion patterns were analyzed for the determination of successful integration.

Detection of the transgene by FISH analysis. Potentially transgenic cells were prepared for FISH analysis as described in Example 2. For GFP sequence detection, pre-labeled hybridization probe (3 μl) was applied for 6 hours at 37° C. and sealed with a cover slip and rubber cement. The hybridization was stopped with 0.4×SSC/0.3% NP-40 at 73° C. and washed again with 2×SSC/0.1% NP-40 to remove all remaining unhybridized probe. The nuclei were counterstained with 5 μg/ml Hoechst 33342 and mounted in Vectashield for observation under conventional epifluorescence and confocal microscopy. Simultaneous FISH was performed for several known rhesus chromosome sequences in order to determine localization of the incorporated transgene. Primers recognizing the X chromosome (Vysis, Downers Grove, Ill.), and sequences on chromosomes 13 and 21 (Vysis, Downers Grove, Ill.) were used.

Karyotype analysis and detection of the transgene by in situ PCR. In situ PCR was performed to amplify a single copy gene sequence in the target cell genome. Instead of using extracted DNA, this technique was performed on cells that were fixed on a slide. DNA primers recognizing the GFP gene were used. Embryonic cells were prepared as described in Example 1. The PCR amplification cycle was 94° C. for 3 minutes followed by thirty cycles of 94° C. for 1 minute, 60° C. for 1 minute, and 72° C. for 1 minute. Fluorescent nucleotides were used for the amplification process and the cells were counterstained with Hoechst 3342. The signal was observed under epifluorescence or enhanced by an additional hybridization step with a specific probe that recognizes the amplicon. The amplicon can be detected in metaphase chromosome spreads as well as in interphase cells. To determine the mosaicism of transgenic embryos, a blastomere from a single embryo was dissociated and used for in situ PCR. In situ PCR can be used to not only determine the presence of the transgene but also the location of the transgene in the genome.

### Example 3

### Retroviral Gene Transfer

Transgenic monkeys expressing GFP were produced by injecting pseudotyped replication-defective retroviral vector into the perivitelline space (PVS) of mature rhesus oocytes, which were later fertilized by intracytoplasmic sperm injection (ICSI). Three healthy males were born from the twenty embryo transfers, and at least one was transgenic.

Vector construction. A 0.75-kb fragment containing the entire coding region of GFP gene was recovered by Hpa I and Hind III digestion of GFP expression vector, pEGFP-N1 (Clontech Laboratories, Inc., Palo Alto, Calif.). The GFP gene fragment was inserted into the Hpa I and Hind III sites of the multiple cloning site in the retrovirus expression vector, pLNCX (Clontech Laboratories) and the GFP gene was regulated by a CMV promoter (plasmid: pLNC-EGFP).

The plasmid phEFnGFP, which contains the hEF-1α promoter and GFP, was digested with Eco RV and Not I followed by filling-in to create a blunt ended site. The 3.44 kb digest fragment was inserted into a blunt-ended site of pLNCX. This second retroviral vector was designated pLNEF-EGFP. The plasmids were stably transfected into the 293 GP packaging cell line and the GFP-expressing cells were sorted by flow cytometry and selection by neomycin (G418). The packaging cell was then transfected with vesicular stomatitis virus envelope glycoprotein G (VSV-G). The supernatant was collected at 48 hours post-transfection and concentrated by ultracentrifugation. The viral titer was determined, and the aliquoted solution was stored at −80° C.

Replication competent retroviris. There is a remote risk of recombination between the vector DNA and the host genomic DNA resulting in the release of viral particles. Thus, to test the safety of this retroviral technology, inoculates were analyzed for replication competent retrovirus (RCR). The assays utilized to analyze for the presence of RCR included the 3T3 amplification assay; the Sarcoma positive, Leukemia negative (S+L-) assay; and PCR analysis of specific retroviral sequences.

The supernatant from packaging cells at the initial vector collection and supernatant from an extended culture (1 week) were collected and submitted for the 3T3 amplification assay followed by the S+L-assay to detect if any RCR is present. If RCR was found in the packaging cell line, then all related products were discarded. If the inoculates were RCR-free or replication incompetent, then the pseudotyped vector was collected by standard collection procedures and used for oocyte injection.

Blood samples were collected from surrogate females before embryo transfer. A total of 4 blood samples were collected from non-pregnant and pregnant females including the pre-embryo transfer, day 30, day 90, and the day of parturition. Additionally, surrogates were tested 6 months post-birth (or post-embryo transfer) to determine their RCR status. Serum or whole blood from these samples were analyzed by CV-1/S+L-assays, PCR, Southern analysis, and retroviral analysis using clonal CV-1-LNC-EGFP cells.

Blood samples were also obtained from egg donors before oocyte aspiration and samples (blood and semen) from semen donors were obtained routinely to use as controls. Samples from the placenta, cord, cord blood, and buccal smear of the infant were obtained at birth, and blood samples were collected at 1, 3, 6, and 12 months of age as well as one skin and muscle biopsy. All samples were analyzed using the CV-1/S-assay, PCR, Southern analysis (when adequate DNA was available), and retroviral analysis using clonal CV-1-LNC-EGFP cells.

For the 3T3 amplification assay, 5% of the tissue culture medium, supernatant, serum, or whole blood was placed on rapidly dividing NIH/3T3 cells (60% confluence) in the presence of 8 mg/ml of polycation for 12 hours at 37° C. Minced tissues and cells derived from potential RCR carriers, such as lymphocytes, were co-cultured with rapidly dividing NIH/3T3 cells (60% confluence) in the presence of 8 mg/ml of polycation for 48 hours at 37° C. At 48 hours post-culture, samples were removed, washed, and replaced with fresh culture medium. Medium was changed on day 4 and a continuous culture was maintained until day 7. On day 7, the supernatant was collected and filtered with a 0.45 mm syringe filter to remove any cell debris. The supernatant was then analyzed using the S+L-assay. The 3T3 amplification assay permits amplification of a small number of RCR. For the CV-1 amplification assay, the rhesus CV-1 cell line was used instead of NIH-3T3.

The S+L-assay utilizes feline PG-4 cells to detect the presence of RCR by the formation of focus formation units (ffu). Supernatant collected from 3T3 amplification assay was placed on rapidly dividing PG-4 cells (60% confluence) in the presence of 8 mg/ml of polycation for 12 hours at 37° C. At 12 hours post-culture, samples were removed, washed and fresh culture medium was added. Fresh medium was replaced every four days and the formation of foci was examined on day seven and fourteen. Each foci was picked and analyzed by PCR to confirm the presence of RCR.

PCR analysis was performed using primer sets designed to amplify specific retrovirus sequences. DNA was extracted from blood, as well as skin and muscle biopsies when blood samples were not adequate. Target sequences includes dVSV-G envelope gene derived from vesicular stomatitis virus, and gag and pol genes of the packaging cells that are derived from MoMLV.

Initial Southern blots were optimized on control rhesus DNA using VSV-G, gag, and pol as probes. Restriction endonucleases were used based on the gag, pol, GFP, and VSV-G sequences. Southern analyses were performed on blood and tissue biopsies from offspring as well as, blood samples from all surrogate females. In addition, Southems were performed to determine if the transgene (i.e., GFP) was integrated into the genome of the offspring genome.

A transduced CV-1 cell line with the retroviral vector that encodes the GFP reporter gene was established. In order to have sufficient GFP signal, LNC-EGFP was used due to its high GFP expression. CV-1 cells were infected with either the pseudotyped retroviral vector or transfected by traditional methods. Transduced cells were selected by neomycin and GFP positive cells were sorted by flow cytometery. Individual cells were sorted into a 96-well plate and clonal CV-1-LNC-EGFP cell lines were established. The CV-1-LNC-EGFP cell replaces the NIH 3T3 amplification process. Serum or samples from exposed animals were used to inoculate the CV-1-LNC-EGFP cell line. Following 1 week of amplification, the supernatant was collected, filtered, and then used to inoculate “traditional” CV-1 cells. The detection of either a GFP-expressing or neomycin-resistant cells indicate the presence of RCR.

Oocyte injection. The perivitelline space (PVS) of mature rhesus oocytes was injected with a high titer (10⁸ to 10⁹ cfu/ml) Moloney retroviral vector pseudotyped with VSV envelope glycoprotein G (VSV-G pseudotype) (Chan et al., 1998). The VSV-G pseudotype carried the GFP gene under the control of the cytomegalovirus early promoter (CMV) [LNCEGFP-(VSV-G)] or the human elongation factor-1 alpha promoter (hEF-1α) [LNEFEGFP-(VSV-G)].

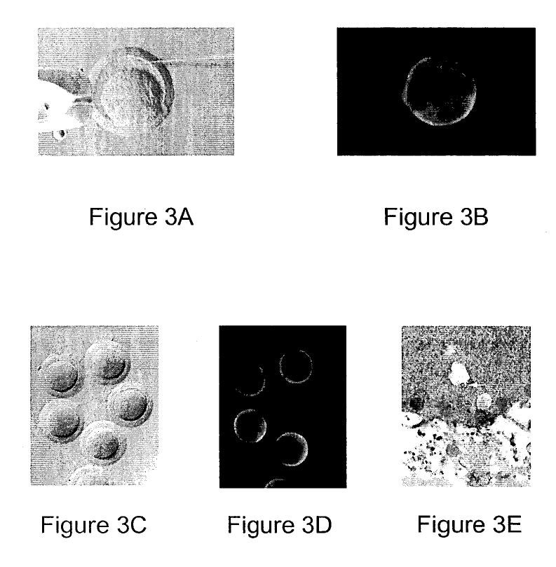

Approximately 10-100 pl were introduced into the PVS of the oocyte; therefore, between 1 and 10 vector particles were introduced using LNCEGFP-(VSV-G)[10⁹ cfu/ml] and between 0.1 to 1 with LNEFEGFP-(VSV-G)[10⁸ cfu/ml] (FIGS. 3A-3D). Oocytes were cultured for 6 hours before fertilization by ICSI. A total of 224 oocytes were injected and 126 oocytes (57%) developed beyond the 4-cell stage and forty embryos (4- to 8-cell stage) were transferred to twenty surrogates, each carrying two embryos. Surrogate females were selected based on serum estradiol and progesterone levels (Hewitson et al., 1998).

The retroviral vector was incorporated into the oocyte in less than 4.5 hours post-PVS injection as imaged by electron microscopy (FIG. 3E). Oocytes were fixed in Ito-Karnovsky's fixative at room temperature for one hour, rinsed in 0.1 M NaCacodylate buffer and post-fixed in 1% OSO₄ with 0.5% K₃Fe(Cn)₆ in 0.1 M NaCacodylate for 1 hour. After rinsing, the oocytes were embedded in agarose blocks for processing. The oocytes were prestained with 4% uranyl acetate stain for 1 hour, rinsed with water, dehydrated with a graded series of acetone, infiltrated with Epon 812, and embedded. Ultrathin sections were cut with a MT5000 ultratome, collected on 300-mesh grids and stained with uranyl acetate and then lead citrate. Sections were viewed with a Philips 300 Electron Microscope and images were recorded on Kodak 4489 negative film.

Five pregnancies resulted in the births of three healthy males, a set of fraternal twins miscarried at 73 days (150-155 days normal gestation), and a blighted pregnancy (Table 1). One fetal twin of the miscarriage was an anatomically normal male, while the other was largely resorbed in utero. The three births and the blighted pregnancy resulted from nine embryo transfers using the LNEFEGFP-(VSV-G), whereas the twin pregnancy was established from eleven embryo transfers using the LNCEGFP-(VSV-G).

TABLE 1
Transgenesis by VSV-G pseudotyped infection in rhesus monkey.

| Construct | pLNC-EGFP | | pLNEF-EGFP | | Total | |
|---|---|---|---|---|---|---|
| Eggs injected with vector | 157 | | 67 | | 224 | |
| Eggs fertilized | 157 | | 65 | | 222 | |
| Fertilization rate | 69% | (108) | 89% | (58) | 75% | (166) |
| Embryonic development | 85 | (54%) | 41 | (63%) | 126 | (57%) |
| Embryos transferred (Two/surrogate) | 22 | | 18 | | 40 | |
| Number of surrogates | 11 | | 9 | | 20 | |
| Pregnancies/surrogate | 1 | (9%) | 4 | (44%) | 5 | (25%) |
| Fetal losses | 2 | (100%) | 1 | (25%) | 3 | (50%) |
| Births | 0 | (0%) | 3 | (17%) | 3 | (50%) |
| Transgenic | 2 | (100%) | 1 | (25%) | 3 | (50%) |

| | | | | | |
|---|---|---|---|---|---|
| Transgenic birth/embryo transfer | 0 | 1 | (5.5%) | 1 | (2.5%) |
| Transgenic birth/pregnancies | 0 | 1 | (25%) | 1 | (20%) |

Detection of transgene integration. To determine transgene integration, transcription, and expression, tissue samples (hair, blood, umbilical cords, placenta, cultured lymphocytes, buccal epithelial cells, and urogenital cells passed in urine) were obtained from the newborns. Tissues from the male stillborn, the resorbed fetus, and the blighted pregnancy were also analyzed. The samples were extracted by DNA extraction as described in Example 1 except blood, buccal epithelial cell, and urine samples. Dried blood spots from a heel stick were spotted onto 3M paper and extracted by an alkaline extraction method. Briefly, a 5 mm disk was punched from each individual dried blood spot, 0.2 M NaOH (20 μl) was added, and samples were incubated at 75° C. for 30 minutes. The extracts were neutralized with 180 μl of 0.02 M Tris-HCl (pH 7.5). An aliqout of the extract (5 μl) was used for PCR reaction (Rudbeck and Dissing, 25 BIOTECHNIQUEs 588, 1998). Bucccal cells were isolated using the MasterAmp Buccal swab DNA extraction kit™ (Epicentre Corp, Madison, Wis.). Urine samples (0.1-0.3 ml) were combined with 5 ml TNE (10 mM Tris-HCl (pH 8.0), 1 mM EDTA, and 100 mM NaCl), centrifuged at 3,000 rpm for 10 minutes, and the pellets were used for DNA extraction (Hayakawa and Takenaka, 48 AM. J. PRIMATOL. 299-304, 1999).

Genomic DNA was analyzed by PCR using a primer set flanking the GFP gene and the vector. For the GFP gene, the 5′ primer (5′-TGAACCGCATCGAGCTGAAG-3′) is located at the GFP gene and the 3′ reverse primer (5′-CTACAGGTGGGGTCTTTCAT-3′) is located at the flanking region of the vector. PCR analysis yielded a 552-bp amplicon from pLNC-EGFP and a 435-bp amplicon from pLNEF-EGFP. For the β-globin internal control, the β-globin 5′ primer (5′ GATGAAGTTGGTGAGGC-3′) and the 3′ reverse primer (5′ ACCCTTGAGGTTGTCCAGGT-3′) were used. This primer set yielded a 318-bp amplicon following amplification of the β-globin gene. For the provirus sequence, the 3′LTR forward primer (5′-ACCTGTAGGTTTGGCAAGCT-3′) located at the U3 region, and the 5′LTR reverse primer (5′-GAAATGAAAGACCCCCGTCG-3′) located at the U5 region of the pLNCX were used for detection. This primer set, 5′LTR and 3′LTR yielded a 500-bp fragment amplicon after amplification of provirus sequence.

The PCR reaction for each primer set was 94° C. for 1 minute, 55° C. for 1 minute, and 72° C. for 1 minute for a total of 35 cycles. An aliquot of the PCR reaction product was analyzed on a 2% agarose gel.

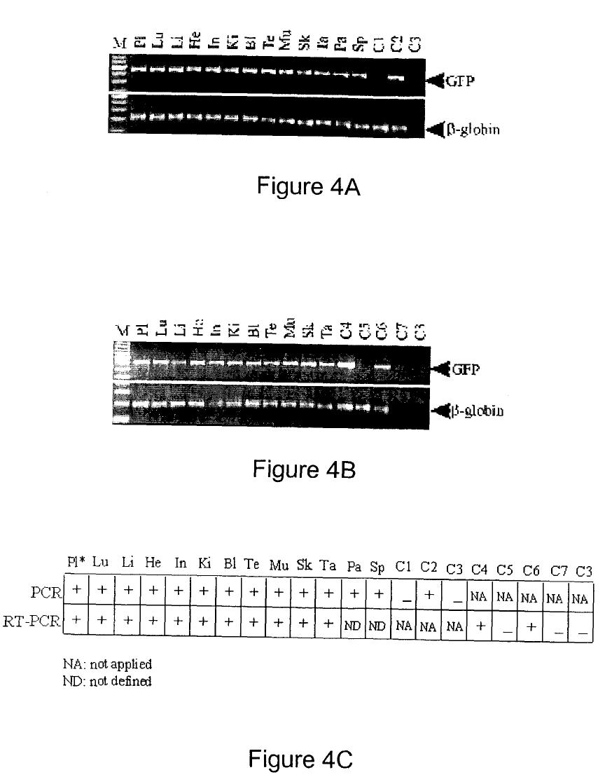

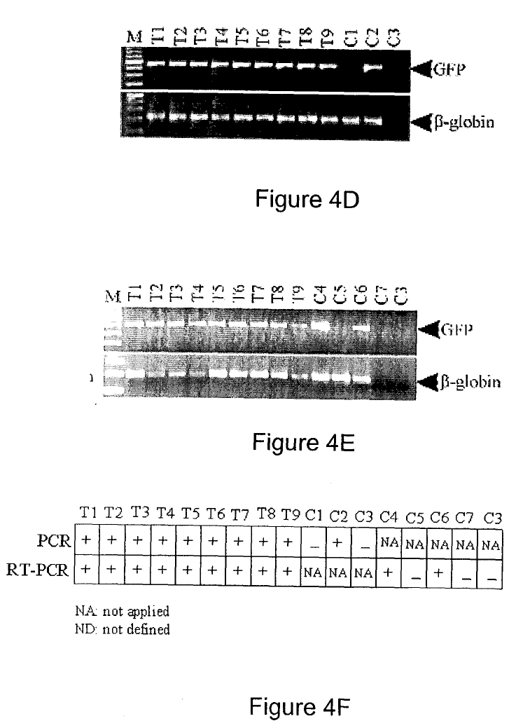

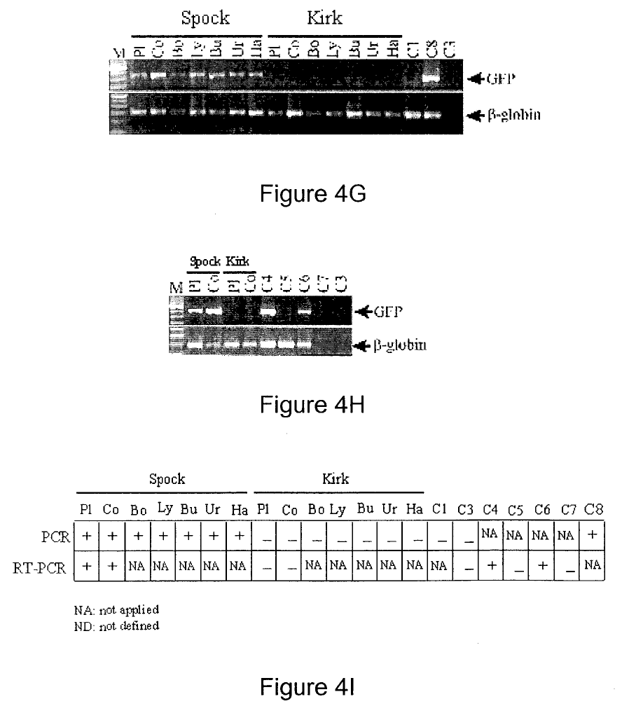

The presence of the GFP transgene was demonstrated in all tissues analyzed from one newborn and the transgene was present in all tissues analyzed from both stillbirths including placenta and testes (FIGS. 4A and 4C).

Detection of GFP expression by RT-PCR. To determine the level of the GFP transcript, RT-PCR was performed using a specific primer set for the transgene. Total RNA (2.5 μg) was prepared and treated with DNase I. The RNA was then reverse transcribed using the RETROscript first-strand synthesis RT-PCR kit™ (Ambion, Austin, Tex.). PCR was performed on the GFP transcript produced by the reverse transcription reaction with the GFP forward primer (5′ ACGGCAAGCTGACCCTGAAG-3′) and the GFP reverse primer (5′ GGGTGCTCAGGTAGTGGTTG-3′). This primer set yielded a 494-bp amplicon following amplification of the GFP cDNA. For the β-globin internal transcript control, the β-globin primer set previously described was used. The rime set yielded a 242-bp amplicon after amplification of β-globin transcript.

Transgene transcription was demonstrated in all tissues from the fetuses and the infant carrying the transgene (FIGS. 4B and 4C), providing confirmation of their transgenic status.

Detection of GFP expression by immunocytochemistry. The expression of GFP was also detected by anti-GFP imaging. Direct fluorescence examination of the stillborn was performed using Nikon SMZ dissecting microscope equipped with fluorescent isothiocyanate (FITC) filters and a Princeton CCD camera (ORCA). Images were captured and analyzed by Metamorph software (Universal Imaging, West Chester, Pa.). Biopsied tissue samples were snap frozen in Tissue-Tek O.C.T. (Sakura Finetek U.S.A., Inc., Torrance, Calif.) and frozen sections (10 μm) were cut using a cryostat. The sections were fixed in 2% paraformaldehyde in 0.05 M PBS for 10 minutes at room temperature, rinsed in PBS, and then blocked in 10% goat serum in PBS for 20 minutes at room temperature on a shaking platform. The primary monoclonal anti-GFP antibody (1:100; Clontech Laboratories) was diluted in PBS with 1.5% goat serum, added to tissue sections, and the sections were incubated for 60 minutes on a shaking platform at room temperature. After an extensive PBS rinse, the GFP primary antibody was detected using rhodamine-conjugated anti-mouse (IgG) secondary antibody (1:50) diluted in PBS with 1.5% goat serum for 45 minutes at room temperature on shaking platform in the dark. After repeated rinses in PBS, the DNA was counterstained with Hoechst 33342 (5 μg/ml) for 2 minutes prior to mounting in Vectashield antifade (Vector Labs, Burlingame, Calif., USA). Slides were examined with Nikon Eclipse epifluorescent microscope equipped with appropriate filters, high numerical aperture objectives, and a digital CCD camera using Metamorph software.

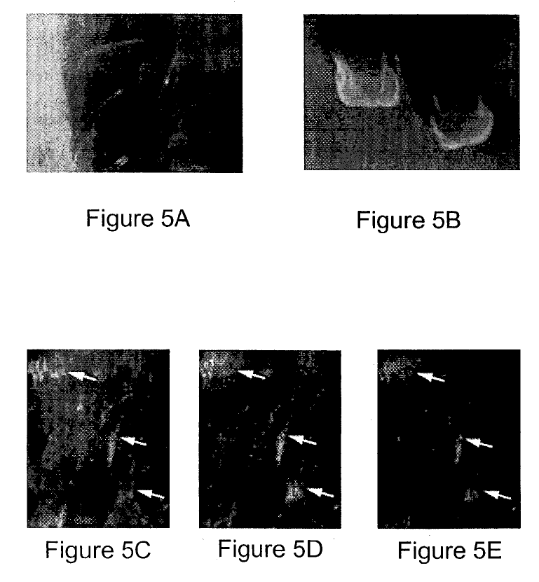

The direct fluorescence of GFP in the hair, toenails, and placenta of a stillborn fetus provided evidence of transgenesis (FIGS. 5A, 5B, and 5D). Immunostaining examination of the frozen tissue sections demonstrated the presence of the GFP protein (FIG. 5C). Overlay of the anti-GFP detection and epifluorescence images demonstrated the co-localization of direct GFP fluorescence with anti-GFP imaging (FIG. 5E). Neither direct nor indirect fluorescence is observed in control fetuses.

Southern blot analysis. Genomic DNA was digested with the restriction enzyme HindIII (single digestion site in pLNC-EGFP). DNA fragments were separated by electrophoresis on a 0.8% agarose gel and transferred to Hybond-N+ nylon membranes. The blot was hybridized with a ³²P-labeled GFP fragment in rapid hybridization buffer (Amersham). After 5 washes at 65° C. with high stringency buffer, the blot was exposed to Phospho Screen (BIO RAD) for 36-48 hours and signal was detected by a Molecular Imager FX (BIO RAD). The blot was then exposed to X-ray film at −80° C. for 7-10 days.

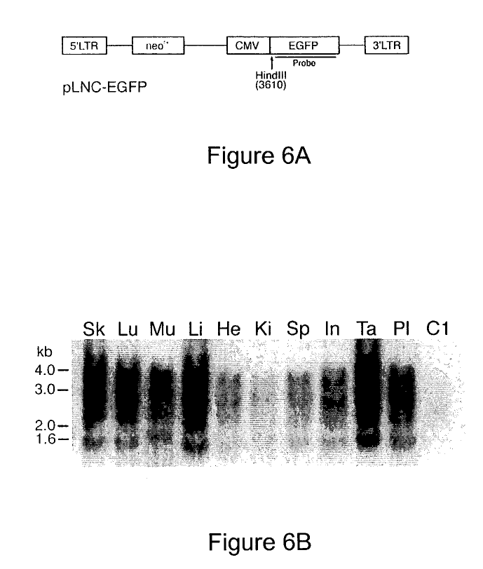

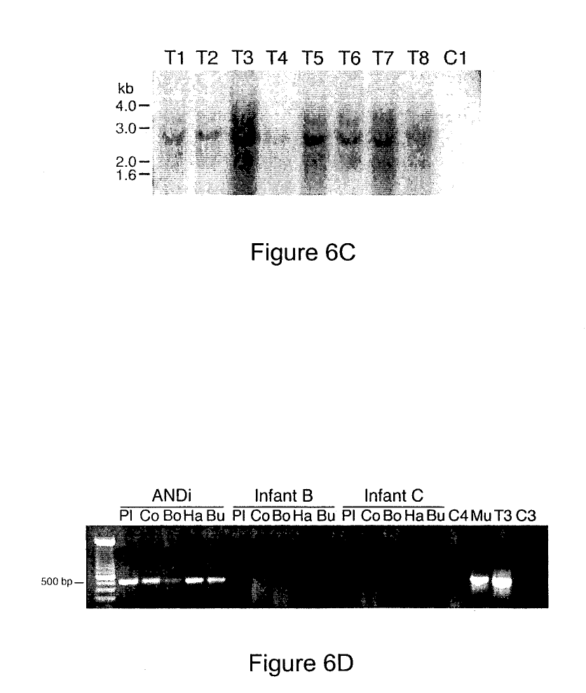

Southern blot analysis of tissues from a stillbirth and resorbed fetus demonstrated multiple integration sites into their genomic DNA (FIGS. 6B and 6C). Vector integration was determined by PCR of placenta, cord, blood, hair, and buccal cells using a primer set specific for the unique retroviral LTR regions indicative of successful provirus integration into the host genome. This provirus sequence was found in one infant (“ANDi”) and both stillbirths (Infant B and C) (FIG. 6D).

### Example 4

### Intracytoplasmic Nuclear Injection (ICNI)

Isolation of cleavage stage blastomeres. Four to sixteen-cell in vitro produced embryos are incubated briefly in Ca²⁺, Mg²⁺-free TALP-HEPES to loosen the association between the blastomeres. The medium is supplemented with 7.5 μg/ml cytochalasin B to relax microfilament network underneath the plasma membrane of blastomeres and consequently increase membrane elasticity. The embryo is held in place by a holding pipette and an enucleation pipette (20-25 μm inner diameter) is inserted through the zona pellucida and individual blastomeres are removed by aspiration (Prather et al., 255 J. EXP. ZOOL. 355-58, 1990; Krisher et al., 78 J. DAIRY SCI. 1282-88, 1995). If necessary, blastomeres are disaggregated by repeated aspiration and expulsion from the pipette. Alternatively, zonae of donor embryos are removed by a short pronase treatment (0.5% pronase for 1.5 minutes), the blastomeres are washed, and placed into Ca²⁺ and Mg²⁺-free medium for 30 minutes. Blastomeres are then dissociated using a glass pipette in the presence of 0.25% trypsin and used for nuclear transfer (Collas and Robl, 43 BIOL. REPROD. 877-84, 1992; Stice and Robl, 39 BIOL. REPROD. 657-664, 1988; Kanka et al., 43 MOL. REPROD. DEV. 135-44, 1996).

Isolation of inner cell mass blastomeres. Inner cell mass cells (ICMs) are isolated from rhesus expanded blastocysts by immunosurgery (Solter and Knowles, 72 PROC. NATL. ACAD. SCI. USA 5099-102, 1975; Keefer et al., 50 BIOL. REPROD. 935-39, 1994). Briefly, the trophectoderm cells are labeled with a rabbit anti-rhesus monkey spleen cell antiserum. After 3 washes in TALP-HEPES, blastocysts are incubated in guinea pig complement, diluted 1:10 in CMRL medium, containing 20 μg/ml propidium iodide (PI) and incubated for 10-15 minutes at 37° C. (Handyside & Hunter, 231 J. EXP. ZOOL. 429-34, 1984; 1988; Hardy et al., 107 DEVELOPMENT 594-604, 1989). This activates the complement cascade rendering the trophectoderm cells permeable to PI. The ICMs, within the lysed trophectoderm cells, are returned to the incubator for 30 minutes, prior to isolation of the ICMs by gentle pipetting. ICM cells are disaggregated in Ca²⁺, Mg²⁺-free TALP for 2 minutes. Individual blastomeres are isolated by repeated pipetting and are cultured singly in TALP medium prior to nuclear transfer.

Isolation of blastomere nuclei. Isolated rhesus blastomeres are induced to exit cell cycle in G0/G1 by serum starvation. The blastomeres are first swollen in a hypotonic solution (0.8% NaCitrate, 0.1% BSA) for 10 minutes at 37° C. before centrifugation through a sucrose gradient at 21,000 g for 20 minutes at 37° C. The centrifugation force tears the cells apart, leaving the nucleus surrounded by a cell membrane and a small amount of cytoplasm (karyoplast). The karyoplasts settle at a density of about 1.3 g/ml sucrose. To remove the sucrose, the karyoplast suspension is washed in embryo culture media. Blastomeres are held on ice until the majority of them show cytoplasmic inclusion but nuclear exclusion of TRITC-IgG. Nuclei are rinsed two times in transport buffer and will be used fresh for ICNI.

Blastomere nucleus injection by ICNI by two-step protocol. Metaphase II-arrested oocytes are first enucleated using standard methods (Dominko et al., 60 BIOL. REPROD. 1496-502, 1999). Holding pipettes are prepared from borosilicate glass capillaries (Sutter Instrument Co., San Rafael, Calif.) with the use of a Flaming Brown horizontal micropipette puller. Injection procedures are performed on a Nikon Diaphot microscope equipped with Hoffman modulation contrast (HMC) optics. The holding pipette is held in a Narishigi (MN-151) manipulator attached to a Hamilton syringe. The injection pipette is mounted in a motorized Eppendorf (5170) micromanipulator attached to a Narishigi (IM-6) injection system. Injections are carried out at 32° C. in 100 μl drops of TALP-HEPES placed in the lid of 100 mm tissue culture dish, covered with light mineral oil (Hewitson et al., 1996). A single blastomere nucleus is aspirated into the injection pipette and then inserted into the oocyte cytoplasm (with the polar body at 12 o'clock) after gentle cytoplasmic aspiration. The blastomere nucleus is deposited in the center of the oocyte and the injection pipette withdrawn.

Blastomere nucleus injection by ICNI by one-step protocol. In this one-step protocol, a single blastomere nucleus is aspirated into the injection pipette and then inserted into the oocyte cytoplasm (with the polar body at 4 o'clock) after gentle cytoplasmic aspiration. The blastomere nucleus is deposited in the center of the oocyte and the injection pipette carefully moved to the meiotic spindle (visualized by epifluorescence illumination). The spindle is removed by gentle aspiration and the injection pipette withdrawn. Confirmation that successful enucleation of the injected oocyte has occurred is performed by the fluorescent analysis of the removed karyoplast.

Chemical activation of oocytes following blastomere ICNI. Chemical activation is induced by a 5-minute pulse of ionomycin (5 mM; CalBiochem), a calcium ionophore, just following blastomere injection or 4-6 hours after blastomere nucleus injection. If this is not sufficient to initiate and sustain activation, a combination of ionomycin and 4 hours in 1.9 mM 6-DMAP are used for activation as described by Susko-Parrish et al., (166 DEV. BIOL. 729-39, 1994).

Sperm cytosolic (oscillin) activation of oocytes following blastomere ICNI Activation of oocytes by a sperm cytosolic factor (oscillin; MW=33 kDa) stimulates repetitive calcium release and initiates cortical granule exocytosis, pronuclear formation, and cleavage events in a number of mammals (Dale et al., 41 EXPERIENTA 1086-70, 1985; Swann, 110 DEVELOPMENT 1295-1302, 1990; Stice and Robl, 39 MOL. REPROD. 657-64, 1990; Parrington et al., 379 NATURE 364-68, 1996). Activation of unfertilized rhesus oocytes with extracts prepared from rhesus sperm are microinjected into ICNI oocytes to initiate similar oocyte activation. Rhesus sperm is collected by penile electroejaculation and washed once in TALP-HEPES culture medium. The sperm pellet is then washed 3 times in an modified intracellular buffer (ICB) composed of 120 mM KCl, 20 mM HEPES, 100 μM EGTA, and 10 mM sodium glycerophosphate, pH 7.5 (Swann, 1990). The final sperm pellet is adjusted to 5-10×10⁸ sperm/ml in ICB and then lysed by 4 freeze-thaw cycles. The lysed samples are centrifuged at 100,000×g for 1 hour at 4° C. and the clear supernatant is collected as the sperm cytosolic fraction. This fraction is concentrated 3-5 fold using Centricon-30 microfiltration membranes (Amicon, Beverly, Mass.), and stored in 10 μl fractions at −80° C. until use. To initiate activation, 8-10 pl of concentrated sperm cytosolic fraction (˜5% of egg volume) is microinjected into ICNI oocytes using micropipettes with 1-2 μm tips. Injected oocytes are returned to culture at 37° C. until transferred to recipient females or fixed for immunocytochemical analysis.

Electrical activation of oocytes following blastomere ICNI Oocytes are placed into fusion medium (0.25 M sorbitol, 100 mM Ca-acetate, 0.1 M Mg-acetate (pH 7.2), 265 mOsm) and allowed to equilibrate for 10 minutes. After equilibration, the oocytes are transferred into a fusion chamber consisting of two parallel wires 500 um apart. The chamber is overlaid with the fusion medium and oocytes are activated by two 20 μsec pulses (2.4 kV field strength) using BTX 2000 electrocell manipulator. Oocytes are washed and placed into embryo culture medium until transferred to recipient females or fixed for immunocytochemical analysis.

Nuclear transfer by fusion using a two-step procedure. In a two-step procedure (Prather et al., 1987, 1990; Stice et al., 38 MOL. REPROD. DEV. 61-68, 1994; Dominko et al., 1999), the oocyte is enucleated and a donor blastomere inserted through the same zona opening immediately following enucleation (enucleation-transfer). The two-step procedure allows for faster production of a nuclear transfer couplet since the enucleated oocyte is held on a holding pipette throughout the procedure and the zona opening made during enucleation can easily be found and used again for the deposition of a blastomere. Since the procedure needs to be performed in the presence of microfilament inhibitors to ensure that the oocyte plasma membrane remains continuous, possible damaging effects of this long exposure to the inhibitors on later embryonic development have to be considered. After nuclear transfer is complete, the nuclear transfer couplets are placed into inhibitor-free medium for recovery prior to fusion. When an average of ten oocytes are used at any given time for nuclear transfer, the duration of enucleation-transfer is not expected to be longer than 30-45 minutes.

Nuclear transfer by fusion using a three-step procedure. In a three-step procedure (Wilmut et al., 385 NATURE 810-13, 1997; Bondioli et al., 33 THERIOGENOLOGY 165-74, 1990; Bames et al., 36 MOL. REPROD. DEV. 33-41, 1993), the oocyte is enucleated, returned to in vitro culture to recover from the procedure, and the donor blastomere inserted 20-30 minutes later, again through the same zona opening (enucleation-recovery-transfer). The three-step procedure performed on the same number of oocytes requires an additional 30 minutes for its completion: 20 minutes for recovery of enucleated oocytes in inhibitor-free medium and 10 minutes for repositioning of the enucleated oocytes such that the zona openings are found and aligned properly for transfer of a blastomere. However, using this approach, the time the oocytes spend in the presence of microfilament inhibitors are shortened and donor cells are never exposed at all. Nuclear transfer units are placed into fusion medium and fused. Prior to fusion nuclear transfer units are aligned, such that the contacting oocyte and blastomere membranes are perpendicular to the electric current. Following fusion the oocytes are washed and placed into embryo culture medium until transferred to recipient females or fixed for immunocytochemical analysis.

Embryo Transfer by Laparotomy. Surgical embryo transfers are performed by mid-ventral laparotomy as described by Wolf et al. (41 BIOL. REPROD. 335-46, 1989). The oviduct is cannulated using a Tomcat catheter containing two 4- to 8-cell stage embryos in HEPES-buffered TALP, containing 3 mg/ml BSA. Embryos are expelled from the catheter in about 0.05 ml of medium while the catheter is withdrawn. The catheter is flushed with medium following removal from the female to ensure that the embryos are successfully transferred. Exogenous progesterone may be administered at the time of embryo transfer and during implantation to help initiate and sustain pregnancy.

Somatic nucleus injection by ICNI. Skin samples are obtained from adult rhesus monkeys by biopsy. Tissue samples are minced and incubated in 0.25% trypsin-EDTA in PBS for 30 minutes with occasional stirring. After 30 minutes, the cell suspension is allowed to sediment for 10 minutes at room temperature and the supernatant containing dissociated cells, removed and placed into a new tube. The sample is centrifuged and washed at least three times in DMEM medium, supplemented with 10% FCS. The final cell pellet is resuspended in 5 ml of the same medium and incubated at 37° C., 5% CO2 in air with maximum humidity. The primary fibroblast culture is passaged when the cells reach confluency (usually once per week). At the time of every passage, a sample of fibroblasts is frozen for DNA analyses. Four to six days prior to nuclear transfer, fibroblasts are cultured in DMEM alone (without serum) in order to induce their accumulation in G0/G1 phase of the cell cycle. The ICNI procedure is performed as described above.

### Example 5

### Pronuclear Injection

Production of Zygotes for Pronuclear Injection. Zygotes are produced by ICSI, as described in Example 1, except that the sperm are not modified with the transgene. Pronucleate zygotes are used for pronuclear injection at approximately 10-15 hours post-ICSI.

Pronuclear Injection. Zygotes are transferred to 100 μl wash medium in a 100 mm petri dish and covered with mineral oil. A holding pipette, with an internal diameter of 20-30 μm, is attached to the Narishigi micromanipulator and connected with a microsyringe filled with silicon oil, whereas the holding pipette is filled with fluorinert (Sigma, St. Louis, Mo.). The injection needles are prepared from capillaries which have a notch along the side of the capillary in order to enhance the capillary action. DNA (4 ng/μl) is microinjected into one of the pronuclei using an Eppendorf Transjector 5426. The parameters for the transjector are set with an injection pressure of 300-500 hpa and a compensation pressure of 15-25 hpa. The length of injection is adjusted by observing the swelling of the pronuclei.

Embryos produced by this technique of direct pronuclear injection are analyzed using the methods as described in Example 2.

### Example 6

### Chimeric Construction

Blastomere dissociation and isolation. Chimeric rhesus embryos are constructed from same-sex blastomeres. Embryos at the 4- to 16-cell stage are used as a source of donor transgenic blastomeres. Following a brief incubation in Ca⁺²-, Mg⁺²-free TALP-HEPES to induce blastomere dissociation, cytochalasin B (7.5 μg/ml) is introduced. The embryo is held in place by a holding pipette, an enucleation pipette (20-25 μm I.D.) is inserted through the zona pellucida and individual blastomeres are removed by aspiration (Prather et al., 1990; Krisher et al., 1995). Alternatively, zonae of donor embryos are removed by a short pronase treatment, and then blastomeres are washed and placed into Ca⁺²- and Mg⁺²-free medium for 30 minutes. Blastomeres are then dissociated using a glass pipette in the presence of 0.25% trypsin and transgenic blastomeres are selected under epifluorescence.

Preparation of GFP-expressing transgenic chimeras. A single non-transgenic blastomere from each embryo is used for a FISH assay to determine the sex of the embryo to be used as the blastomere donor. Only transgenic blastomeres selected under fluorescence and originating from the same-sex embryos are then placed into empty zona pellucidae and the same stage embryos are recreated. After aggregation, embryos are cultured in vitro and their development ability determined. The remaining same-sex non-transgenic blastomeres are pooled and control embryos are created in the same way. An alternative is to transfer a transgenic blastomere into a non-transgenic embryo. Since only one blastomere in the newly created embryo will be potentially transgenic, accurate cell lineage of different tissues can be determined.

Embryo biopsy and detection of X and Y chromosomes by FISH analysis in blastomeres. Single blastomeres are isolated by biopsy and processed for FISH. The blastomeres are pipetted onto a slide, the PBS is exchanged with 0.01 N HCl/0.1% Tween-20 to dissolve the zonae and permeabilize cell membranes. The slides are washed in PBS, and dehydrated through an ascending ethanol series. A 20-minute incubation in 100 μg/ml pepsin in 0.01 N HCl at 37° C. allows access to the nuclei for hybridization and removes any cytoplasmic remnants. Prior to hybridization, the slides are dehydrated through another ascending ethanol series (Coonen et al., 9 HUM. REPROD. 533-37, 1994) and then immersed in a denaturing solution (formamide/SSC) for 5 minutes at 73° C. Following the denaturation step, 3 μl of hybridization probe (Vysis: CEP X SpectrumGreen™/CEP Y SpectrumOrange™) is applied for 6 hours at 37° C. The hybridization is stopped with 0.4×SSC/0.3% NP-40 and the slides are then washed with 2×SSC/0.1% NP-40 to remove unhybridized probe. The nuclei are counterstained with 5 μg/ml Hoechst 33342 and mounted in Vectashield. The X and Y chromosomes are detected using conventional and confocal microscopy.

Data analysis. The FISH analysis can be completed within 60 minutes after isolation of embryonic blastomeres and this delay does not have a detrimental effect on aggregation chimeras. Effect of disaggregation and reaggregation on the viability of newly created chimeras are compared with the viability of non-manipulated controls. The chimeras are placed in culture and their development monitored. Embryo development is evaluated by total cell numbers.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

We claim:

1. A method for producing a transgenic animal comprising the steps of:
transferring transgenic embryos, which were produced by transferring exogenous DNA from spermatozoa, to oocytes by intracytoplasmic sperm injection and cultured to an embryonic stage, to the oviducts of surrogate females; and
producing a transgenic animal by parturition.

2. The method of claim 1, wherein said animal is selected from the group consisting of mammals, birds, reptiles, amphibians, and fish.

3. The method of claim 2, wherein said animal is a nonhuman primate.

4. The method of claim 3, wherein said nonhuman primate is selected from the group consisting of rhesus macaque, baboon, capuchin, chimpanzee, pigtail macaque, sooty mangabey, squirrel monkey, orangutan.

5. The method of claim 1, wherein said exogenous DNA is an expression vector.

6. The method of claim 5, wherein said expression vector comprises regulatory nucleic acid sequences and a structural gene sequence.

7. The method of claim 5, wherein said expression vector is selected from the group consisting of plasmid vectors, viral vectors, and retroviral vectors.

8. The method of claim 6, wherein said regulatory nucleic acid sequence is a promoter.

9. The method of claim 8, wherein said promoter is a viral promoter.

10. The method of claim 9, wherein said viral promoter is the cytomegalovirus promoter.

11. The method of claim 8, wherein said promoter is the protamine-1 promoter.

12. The method of claim 8, wherein said promoter is selected from the group consisting of inducible promoter and constitutive promoter.

13. The method of claim 6, wherein said structural gene sequence encodes a polypeptide selected from the group consisting of receptors, enzymes, cytokines, hormones, growth factors, immunoglobulins, cell cycle proteins, cell signaling proteins, membrane proteins, and cytoskeletal proteins.

14. The method of claim 6, wherein said structural gene sequence is a reporter gene.

15. The method of claim 14, wherein said reporter gene is green fluorescent protein gene.

16. The method of claim 14, wherein said reporter gene is selected from the group consisting of β-galactosidase gene, secreted placental alkaline phosphatase gene, and luciferase gene.

17. The method of claim 14, wherein said reporter gene is used to monitor the development of a cell or tissue in a transgenic embryo.

18. The method of claim 6, wherein said structural gene sequence is a disease gene.

19. The method of claim 18, wherein said disease gene is linked to a disease selected from the group consisting of cardiovascular diseases, neurological diseases, reproductive disorders, cancers, eye diseases, endocrine disorders, pulmonary diseases, metabolic disorders, hereditary diseases, autoimmune disorders, and aging.

20. The method of claim 1, wherein said exogenous DNA is labeled with a fluorophore.

21. The method of claim 20, wherein said fluorophore is rhodamine.

22. The method of claim 1, wherein said exogenous DNA consists of one or more expression vectors.

23. The method of claim 5, wherein said expression vector comprises regulatory nucleic acid sequences and two or more structural gene sequences.

24. The method of claim 1, wherein said oocyte is cultured to the 3-16 cell embryo stage.

25. The method of claim 1, wherein said transgenic animal is a model for human disease.

26. The method of claim 25, wherein said human disease is selected from the group consisting of cardiovascular diseases, neurological diseases, reproductive disorders, cancers, eye diseases, endocrine disorders, pulmonary diseases, metabolic disorders, autoimmune disorders, and aging.

27. The method of claim 1, wherein said transgenic animal is a model for hereditary disease.

28. The method of claim 1, wherein said transgenic animal is a model for embryo and fetal development.

29. The method of claim 1, wherein said transgenic animal is model to demonstrate the safety and efficacy of treatments selected from the group comprising drug therapy, gene therapy, stem cell therapy, and somatic cell therapy.

30. The method of claim 1, wherein said transgenic animal is model for disease diagnosis.

31. The method of claim 1, wherein said method is used to preserve an endangered species.

32. The method of claim 1, wherein said method is used for sperm-mediated gene therapy.

33. The method of claim 1, wherein the said spermatazoa are subjected to sanitizing treatments selected from the group consisting of chemical decontamination and physical removal.

34. The method of claim 33, wherein said chemical decontamination is selected from the group consisting of proteinases, DNases, and RNases.

35. The method of claim 33, wherein said sanitizing treatment by physical removal is selected from the group consisting of polystyrene and magnetic beads.

36. A transgenic embryo produced according to the method of claim 1.

37. The transgenic embryo of claim 36, wherein said transgenic embryo is a model for embryo and fetal development.

38. A transgenic animal produced according to the method of claim 1.

39. The transgenic animal of claim 38, wherein said transgenic animal is a model for human disease.

40. The transgenic animal of claim 38, wherein said transgenic animal is a model for hereditary disease.

41. The transgenic animal of claim 38, wherein said transgenic animal is model to demonstrate the safety and efficacy of treatments selected from the group consisting of drug therapy, gene therapy, stem cell therapy, and somatic cell therapy.

42. The transgenic animal of claim 38, wherein said transgenic animal is model for disease diagnosis.

43. The transgenic embryo of claim 36, wherein said transgenic embryo is a transgenic chimeric embryo.

44. A method for producing a transgenic animal comprising the steps of:
transferring transgenic embryos, which were produced by transferring exogenous DNA to oocytes by injection of a retroviral vector, fertilizing the oocytes by intracytoplasmic sperm injection, and culturing said oocytes to the embryonic stage, to the oviducts of surrogate females; and
producing a transgenic animal by parturition.

45. The method of claim 44, wherein said animal is a nonhuman primate.

46. The method of claim 45, wherein nonhuman primate is selected from the group consisting of rhesus macaque, baboon, capuchin, chimpanzee, pigtail macaque, sooty mangabey, squirrel monkey, orangutan.

47. The method of claim 44, wherein said oocyte comprises a prematuration oocyte.

48. The method of claim 44, wherein said oocyte comprises a prefertilization oocyte.

49. The method of claim 44, wherein the perivitelline space of said oocyte is injected with a retroviral vector.

50. The method of claim 44, wherein said oocyte is cultured to the 4-8 cell embryo stage.

51. The method of claim 44, wherein said retroviral vector comprises regulatory gene sequences and structural gene sequences.

52. The method of claim 51, wherein said regulatory sequence is a promoter.

53. The method of claim 52, wherein said promoter is a viral promoter.

54. The method of claim 53, wherein said viral promoter is the cytomegalovirus promoter.

55. The method of claim 52, wherein said promoter is a human elongation factor-1 alpha promoter.

56. The method of claim 44, wherein said retroviral vector is selected from the group consisting of Moloney murine leukemia virus, Harvey murine sarcoma virus, murine mammary tumor virus, and Rous sarcoma virus.

57. The method of claim 44, wherein detection of said retroviral vector is determined by a retroviral assay selected from the group consisting of CV-1/S+L-assay, PCR, Southern analysis, and clonal CV-1-LNC-EGFP cells.

58. The method of claim 44, wherein said retroviral vector comprises a membrane-associated protein.

59. The method of claim 58, wherein said membrane-associated protein is a glycoprotein selected from Rhabdoviridae.

60. The method of claim 59, wherein said glycoprotein is obtained from the group consisting of vesicular stomatitis virus, Piry virus, Chandipura virus, Spring viremia of carp virus, Rabies virus, and Mokola virus.

61. The method of claim 51, wherein said structural gene sequence is a reporter gene.

62. The method of claim 61, wherein said reporter gene is green fluorescent protein gene.

63. The method of claim 61, wherein said reporter gene is selected from the group consisting of β-galactosidase gene, secreted placental alkaline phosphatase gene, and luciferase gene.

64. The method of claim 51, said structural gene sequence encodes a polypeptide selected from the group comprising receptors, enzymes, cytokines, hormones, growth factors, immunoglobulins, cell cycle proteins, cell signaling proteins, membrane proteins, and cytoskeletal proteins.

65. A method for producing a transgenic primate comprising the steps of:
transferring said transgenic embryos which were produced by dissociating blastomeres from an embryo, removing the nuclei from the blastomeres, injecting the blastomere nuclei into enucleated oocytes by intracytoplasmic nuclear injection, and activating and culturing oocytes to an embryonic stage, to the oviducts of surrogate females; and
producing a transgenic primate by parturition.

66. The method of claim 65, wherein said primate is a nonhuman primate.

67. The method of claim 66, wherein nonhuman primate is selected from the group consisting of rhesus macaque, baboon, capuchin, chimpanzee, pigtail macaque, sooty mangabey, squirrel monkey, orangutan, and members thereof.

68. The method of claim 65, wherein inner cell mass cells are isolated from said blastomere for nuclear transfer.

69. The method of claim 65, wherein said oocyte activation is selected from the group consisting of chemical activation, sperm cytosolic (oscillin) activation, and electrical activation.

70. A method for producing a transgenic primate comprising the steps of:
transferring transgenic embryos, which were produced by isolating nuclei from somatic cells, injecting the nuclei into enucleated oocytes by intracytoplasmic nuclear injection, then activating and culturing the oocytes to an embryo stage, to the oviduct of surrogate females; and
producing a transgenic primate by parturition.

71. The method of claim 70, wherein said primate is a nonhuman primate.

72. The method of claim 71, wherein nonhuman primate is selected from the group consisting of rhesus monkey, baboon, capuchin, chimpanzee, pigtail macaque, sooty mangabey, squirrel monkey, orangutan.

73. The method of claim 70, wherein said somatic cells are skin cells.

74. The method of claim 74, wherein said oocyte activation is selected from the group comprising chemical activation, sperm cytosolic (oscillin) activation, and electrical activation.

75. A method for producing a transgenic primate comprising the steps of:
transferring transgenic embryos, which were produced by fertilizing an oocyte by intracytoplasmic sperm injection, then transferring exogenous DNA to the pronucleus of fertilized oocyte by pronuclear injection, then culturing the fertilized oocyte to an embryonic stage, to oviduct of surrogate females; and
producing a transgenic primate by parturition.

76. A method of producing transgenic primate cells wherein said transgenic cells are used to treat human diseases.

77. The method of claim 76, wherein said transgenic primate cells are produced by methods selected from the group consisting of intracytoplasmic sperm injection, retroviral gene transfer, intracytoplasmic nuclear injection, and pronuclear injection.

78. The method of claim 76, wherein said human disease is selected from the group consisting of cardiovascular disease, neurological diseases, reproductive disorders, cancer, eye diseases, endocrine disorders, pulmonary disease, metabolic disorders, autoimmune disorders, and aging.

79. The method of claim 1, wherein said exogenous DNA is bound to spermatozoa comprising the steps of:
mixing exogenous DNA with spermatozoa;
incubating DNA-spermatozoa mixture for 30 minutes at 37° C.; and
washing DNA-bound spermatozoa in TALP-HEPES buffer.
